(19) United States
(12) Patent Application Publication
(10) Pub. No.: US 2014/0212409 A1
(43) Pub. Date: Jul. 31, 2014

(54) METHOD FOR INCREASING DEPOSITION OF COMPLEMENT C3b ON BACTERIAL SURFACE AND PHAGOCYTOSIS BY PHAGOCYTE AND A THERAPEUTIC METHOD AND A THERAPEUTIC AGENT FOR BACTERIAL INFECTIONS

(71) Applicant: KYOWA HAKKO KIRIN CO., LTD., Tokyo (JP)

(72) Inventors: Sascha KRISTIAN, La Jolla, CA (US); Mami KOYAMA, La Jolla, CA (US); Yutaka KANDA, La Jolla, CA (US); Tomoyuki TAHARA, La Jolla, CA (US)

(73) Assignee: KYOWA HAKKO KIRIN CO., LTD., Tokyo (JP)

(21) Appl. No.: 13/965,347

(22) Filed: Aug. 13, 2013

Related U.S. Application Data
(60) Provisional application No. 61/682,404, filed on Aug. 13, 2012.

(51) Int. Cl. C07K 16/12

(52) U.S. Cl. CPC C07K 16/1271; C07K 16/1275
USPC 424/133.1

(57) ABSTRACT

The present invention relates to a method for increasing deposition of complement C3b on the bacterial surface and phagocytosis by phagocytes, a therapeutic method and a therapeutic agent for bacterial infections, using an antibody binding to a molecule on the bacterial surface, in which the antibody is modified by substituting at least one amino acid residue with other amino acid so as to show more enhanced complement-dependent cytotoxicity (CDC) than the antibody before substitution of the amino acid residue.

### TECHNICAL FIELD

The present invention relates to a method for increasing deposition of complement C3b on the bacterial surface and phagocytosis by phagocytes using an antibody binding to a molecule on the bacterial surface, in which the antibody is modified by substituting at least one amino acid residue with other amino acid so as to show more enhanced complement-dependent cytotoxicity (CDC) than the antibody before substitution of the amino acid residue, and a therapeutic method and a therapeutic agent for bacterial infections, characterized in that the antibody binding to the molecule on the bacterial surface, which is modified by substituting at least one amino acid residue with other amino acid so as to show more enhanced CDC than the antibody before substitution of the amino acid residue, is used to increase deposition of complement C3b on the bacterial surface and phagocytosis by phagocytes, thereby reducing bacterial proliferation.

### BACKGROUND ART

Biological defense response such as complement-mediated lysis (CML) or CDC, and phagocytosis via a series of activation cascades by complement component in the blood are known to be important in the treatment of bacterial infections.

Further, the classical complement pathway initiated by binding of C1q to the antibody, the second pathway initiated by cleavage of complement C3, and the lectin pathway initiated by binding of lectin are known as the complement activation cascades. Especially, Gram-positive bacteria are known to be less susceptible to cell damage by complement activity, because they have rigid proteoglycan cell membrane. Phagocytes (granulocytes, macrophages, dendritic cells, and the like) involved in phagocytosis in the body are known to have opsonophagocytosis of ingesting and killing bacteria bound with a complement cascade intermediate, C3b or C3d.

An antibody is a heterotetramer composed of heavy chains (hereinafter, referred to as H chain) and light chains (hereinafter, referred to as L chain), and consists of Fab involved in antigen binding and Fc region binding to Fc receptor (hereinafter, referred to as only Fc). The Fc of antibody is known to mediate CDC, antibody-dependent cellular cytotoxicity (hereinafter, referred to as ADCC), and phagocytosis. It has been known that CDC and/or ADCC activity of antibody can be controlled by substitution of amino acid residues of the antibody Fc.

Further, it has been known that ADCC activity of antibody can be controlled by controlling an amount of fucose which is bound in α-1,6 linkage to N-acetylglucosamine (GlcNAc) present in a reducing end of a complex type N-linked sugar chain which is bound to asparagine (Asn) at position 297 of the antibody Fc region according to EU index (Non-Patent Document 1).

### CITATION LIST

### Non-Patent Document

### Non-Patent Document 1

Kabat et al, Sequence of Proteins of Immunological Interests, 5th edition, 1991

### SUMMARY OF TIE INVENTION

### Problems to be Solved by the Invention

For the treatment of bacterial infections, there is a need for a therapeutic method for increasing antibacterial activity via complement-mediated lysis (or complement-dependent cytotoxicity) or phagocytosis.

### Means for Solving the Problems

The present invention relates to (1) to (11) below.

(1) A method for increasing deposition of complement C3b on the bacterial surface and phagocytosis by phagocytes using an antibody binding to a molecule on the bacterial surface, wherein the antibody is modified by substituting one or more amino acid residues with other amino acids so as to show more enhanced complement-dependent cytotoxicity than the antibody before substitution of the amino acid residues.
(2) The method described in (1) above, wherein one or more amino acid residues are included in the CH2 domain of the antibody Fc region.
(3) The method described in (1) or (2) above, wherein the modified antibody shows more enhanced complement C1q-binding activity than the antibody having an amino acid sequence before substitution of the amino acid residue.
(4) The method described in any one of (1) to (3) above, wherein the subclass of the antibody binding to a molecule on the bacterial surface is human IgG1.
(5) The method described in any one of (1) to (4) above, wherein the bacteria are one or more bacteria selected from Gram-positive and Gram-negative bacteria.
(6) The method described in (5) above, wherein the Gram-positive bacteria are one or more Gram-positive bacteria selected from Bacillus, Listeria, Staphylococcus, Streptococcus, Enterococcus, Clostridium, and Mycobacterium.
(7) The method described in (5) above, wherein the Gram-negative bacteria are one or more Gram-negative bacteria selected from Pseudomonas, Escherichia, Salmonella and Acinetobacter.
(8) The method described in any one of (1) to (7) above, wherein the molecule on the bacterial surface is one or more molecules selected from ganglioside, capsular polysaccharide (CP), surface protein (SP) and lipopolysaccharide (LPS).
(9) The method described in any one of (1) to (8) above, wherein the modified antibody has a complex type N-linked sugar chain in the Fc region, and 20% or more of the total complex type N-linked sugar chain binding to the Fc region is the sugar chain having no α1,6-fucose bound to N-acetylglucosamine at the reducing end of the sugar chain.
(10) A therapeutic agent for bacterial infections, characterized in that an antibody binding to a molecule on the bacterial surface, which is modified by substituting one or more amino acid residues with other amino acids so as to show more enhanced CDC than the antibody before substitution of the amino acid residues, is used to increase deposition of complement C3b on the bacterial surface and phagocytosis by phagocytes, thereby reducing bacterial proliferation.
(11) A therapeutic method for bacterial infections, characterized in that an antibody binding to a molecule on the bacterial surface, which is modified by substituting one or more amino acid residues with other amino acids so as to show more enhanced CDC than the antibody before substitution of the amino acid residues, is used to increase deposition of complement C3b on the bacterial surface and phagocytosis by phagocytes, thereby reducing bacterial proliferation.

### Effect of the Invention

According to the present invention, provided are a method for increasing deposition of complement C3b on the bacterial surface using an antibody binding to a molecule on the bacterial surface, in which the antibody is modified by substituting at least one amino acid residue with other amino acid so as to show more enhanced CDC than the antibody before substitution of the amino acid residue, and a therapeutic method and a therapeutic agent for bacterial infections, characterized in that the antibody binding to the molecule on the bacterial surface, which is modified by substituting at least one amino acid residue with other amino acid so as to show more enhanced CDC than the antibody before substitution of the amino acid residue, is used to increase deposition of complement C3b on the bacterial surface and phagocytosis by phagocytes, thereby reducing bacterial proliferation.

### BRIEF DESCRIPTION OF THE DRAWINGS

FIG. 1 shows the binding activity of anti-ganglioside GD3 antibody for FcγRIA and FcγRIIB.

FIG. 2 shows a method for measuring phagocytosis in vitro using anti-ganglioside GD3 antibody.

FIG. 3 is a histogram showing anti-ganglioside GD3 antibody-dependent ingestion of Staphylococcus aureus by neutrophils.

In FIG. 4, the vertical axis represents the ratio of neutrophils showing anti-ganglioside GD3 antibody-dependent ingestion of Staphylococcus aureus to the total amount of neutrophils, and the horizontal axis represents the antibody concentration.

In FIG. 5, the vertical axis represents the total amount (×10⁶ cells) of Staphylococcus aureus ingested by neutrophils in an anti-ganglioside GD3 antibody-dependent manner, and the horizontal axis represents the antibody concentration.

FIG. 6 shows a method of assay for complement C3b deposition mediated by anti-ganglioside GD3 antibody.

FIG. 7 shows the effect of anti-ganglioside GD3 antibody on complement C3b deposition on Staphylococcus aureus SA113 (ATCC35556), in which the vertical axis represents the mean fluorescence intensity (MFI) of C3b (deposition amount), and the horizontal axis represents the antibody used.

FIG. 8 shows the effect of anti-ganglioside GD3 antibody on C3b deposition on Staphylococcus aureus Newman (ATCC 25905), in which the vertical axis represents the mean fluorescence intensity (MFI) of C3b (deposition amount), and the horizontal axis represents the antibody used.

FIG. 9 shows a method for evaluating the anti-ganglioside GD3 antibody-mediated opsonophagocytosis.

FIG. 10 shows the anti-ganglioside GD3 antibody-mediated opsonophagocytosis of human neutrophils on Staphylococcus aureus SA113, in which the vertical axis represents colony forming unit (CFU)/mL, and the horizontal axis represents the presence or absence of human polymorphonuclear neutrophils (hereinafter, abbreviated to PMN).

FIG. 11 shows the anti-ganglioside GD3 antibody-mediated opsonophagocytosis of human neutrophils on Staphylococcus aureus Newman, in which the vertical axis represents colony forming unit (CFU)/mL, and the horizontal axis represents the presence or absence of PMN and the antibody concentration.

FIG. 12 shows a method of assay for complement C3b deposition by anti-CP5 antibody.

FIG. 13 shows the effect of anti-CP5 antibody on complement C3b deposition on Staphylococcus aureus Lowenstein, in which the vertical axis represents the mean fluorescence intensity (MFI) of C3b (deposition amount), and the horizontal axis represents the type of antibody.

FIG. 14 shows the effect of anti-CP5 antibody on complement C3b deposition on Staphylococcus aureus Lowenstein in the presence or absence of C1q, in which the vertical axis represents the mean fluorescence intensity (MFI) of C3b (deposition amount), and the horizontal axis represents the type of antibody.

FIG. 15 shows the temporal effect of anti-CP5 antibody on complement C3b deposition on Staphylococcus aureus Lowenstein, in which the vertical axis represents the mean fluorescence intensity (MFI) of C3b (deposition amount), and the horizontal axis represents the type of antibody and time (min) after addition of the antibody.

FIG. 16 shows anti-CP5 antibody-dependent ingestion effect of Staphylococcus aureus Lowenstein by neutrophils, in which the vertical axis represents the ratio (%) of neutrophils showing ingestion of Staphylococcus aureus to the total amount of neutrophils, and the horizontal axis represents the antibody concentration.

FIG. 17 shows anti-PspA antibody-dependent ingestion effect of Streptococcus pneumoniac D39 by neutrophils, in which the vertical axis represents the ratio (%) of neutrophils showing ingestion of Streptococcus pneumonia to the total amount of neutrophils, and the horizontal axis represents the type of antibody.

FIG. 18 shows a method of assay for complement C3b deposition by anti-PspA antibody.

FIG. 19 shows the effects depending anti-PspA antibodies, 140G1 and 140H1 on C3b deposition on Streptococcus pneumoniae BAA-658, in which the vertical axis represents the number of cells, and the horizontal axis represents the mean fluorescence intensity (MFI) of C3b (deposition amount).

FIG. 20 shows the effect of anti-PspA antibody, 140H1 on C3b deposition on three Streptococcus pneumoniae strains, PJ-1324, WU2, and BAA-658.

FIG. 21 shows the therapeutic effect of anti-CP5 antibody in the Rowett Nude (hereinafter, referred to as RNU) rat model infected with Staphylococcus aureus MSSA Reynolds (ATCC-25923), in which the vertical axis represents the survival rate (%) of rats in each group, and the horizontal axis represents the day after administration of the bacteria pre-opsonized with each antibody.

### EMBODIMENTS FOR CARRYING OUT THE INVENTION

The present invention relates to a method for increasing deposition of complement C3b on the bacterial surface and phagocytosis by phagocytes using an antibody binding to a molecule on the bacterial surface, in which the antibody is modified by substituting at least one amino acid residue with other amino acid so as to show more enhanced CDC than the antibody before substitution of the amino acid residue.

Further, the present invention relates to a therapeutic method and a therapeutic agent for bacterial infections, characterized in that the antibody binding to the molecule on the bacterial surface, which is modified by substituting at least one amino acid residue so as to show more enhanced CDC than the antibody before substitution of the amino acid residue, is used to increase deposition of complement C3b on the bacterial surface and phagocytosis by phagocytes, thereby reducing bacterial proliferation.

The modified antibody used in the present invention may be any modified antibody, as long as the antibody binds to the molecule on the bacterial surface, in which the antibody is modified by substituting at least one amino acid residue with other amino acid residues so as to show more enhanced CDC. The amino acid residue to be substituted is preferably the amino acid residue in CH2 and CH3 domains (Fragment, crystallizable, hereinafter, referred to as Fc region or Fc) in the antibody constant region, and more preferably at least one amino acid residue included in the CH2 domain.

Examples of the modified antibody used in the present invention may include an antibody binding to a molecule on the bacterial surface, in which the antibody includes an amino acid sequence represented by SEQ ID NO. 1 or 2 in its Fc region.

Examples of the modified antibody used in the present invention may include an antibody binding to a molecule on the bacterial surface, in which the antibody is modified by substituting at least one amino acid residue with other amino acid residue so as to show more enhanced CDC activity and complement C1q-binding activity than the antibody before substitution of the amino acid residue.

Further, in the present invention, the species and subclass of the antibody binding to a molecule on the bacterial surface are not particularly limited, but it is preferably human IgG, and more preferably human IgG1.

The present invention includes a method for increasing deposition of complement C3b on the bacterial surface and phagocytosis by phagocytes using an antibody binding to a molecule on the bacterial surface, in which the antibody is modified by substituting at least one amino acid residue so as to show more enhanced CDC than the antibody before substitution of the amino acid residue, and the modified antibody has a complex type N-linked sugar chain in the Fc region, and 20% or more of the total complex type N-linked sugar chain binding to the Fc region is the sugar chain having no α1,6-fucose bound to N-acetylglucosamine at the reducing end of the sugar chain.

Further, the present invention includes a therapeutic method and a therapeutic agent for bacterial infections, characterized in that the antibody binding to the molecule on the bacterial surface, which is modified by substituting at least one amino acid residue so as to show more enhanced CDC than the antibody before substitution of the amino acid residue and the modified antibody has a complex type N-linked sugar chain in the Fc region, and 20% or more of the total complex type N-linked sugar chain binding to the Fc region is the sugar chain having no α1,6-fucose bound to N-acetylglucosamine at the reducing end of the sugar chain, is used to increase deposition of complement C3b on the bacterial surface and phagocytosis by phagocytes, thereby reducing bacterial proliferation.

The antibody binding to a molecule on the bacterial surface, in which the antibody is modified by substituting at least one amino acid residue so as to show more enhanced CDC than the antibody before substitution of the amino acid residue and the modified antibody has a complex type N-linked sugar chain in the Fc region, and 20% or more of the total complex type N-linked sugar chain binding to the Fc region is the sugar chain having no α1,6-fucose bound to N-acetylglucosamine at the reducing end of the sugar chain, is able to increase deposition of complement C3b on the bacterial surface and has increased Fc receptor-binding activity, thereby increasing phagocytosis by phagocytes.

Meanwhile, “the modified antibody has a complex type N-linked sugar chain in the Fc region, and 20% or more of the total complex type N-linked sugar chain binding to the Fc region is the sugar chain having no α1,6-fucose bound to N-acetylglucosamine at the reducing end of the sugar chain” means that the modified antibody has a complex type N-glycoside linked sugar chain in the Fc region, and 20% or more of the total complex type N-glycoside linked sugar chain binding to the Fc region is the sugar chain having no fucose bound to N-acetylglucosamine at the reducing end of the sugar chain.

The antibody used in the present invention may be, for example, any one of a monoclonal antibody and a polyclonal antibody, and preferably, a monoclonal antibody binding to a single epitope.

The monoclonal antibody may be any one of monoclonal antibodies produced from hybridomas and genetically recombinant antibodies produced by a genetic recombination technology. To reduce immunogenicity in human, a human chimeric antibody (hereinafter, also simply called chimeric antibody), a humanized antibody [also called human complementarity determining region (CDR)-grafted antibody], and a human antibody are preferably used.

In the present invention, the monoclonal antibody is an antibody secreted by antibody-producing cells of a single clone. The monoclonal antibody recognizes only a single epitope (also called antigenic determinant), and the amino acid sequence (primary structure) constituting the monoclonal antibody is uniform.

Examples of the epitope may include a single amino acid sequence, a conformation composed of the amino acid sequence, an amino acid sequence bound with a modification residue such as a sugar chain, a glycolipid, a lipopolysaccharide, an amino group, a carboxyl group, phosphate, sulfate or the like, and a conformation composed of the amino acid sequence bound with the modification residue recognized and bound by a monoclonal antibody. The conformation is a naturally existing three-dimensional structure of a protein, and it refers to a conformation composed of proteins expressed within cells or on cell membrane.

In the present invention, the antibody molecule is also called immunoglobulin (hereinafter referred to as Ig) and human antibody is classified into the isotypes of IgA1, IgA2, IgD, IgE, IgG1, IgG2, IgG3, IgG4 and IgM, based on the difference of molecular structure. IgG1, IgG2, IgG3 and IgG4 having relatively high homology in amino acid sequences are genetically called IgG.

The antibody molecule is composed of polypeptides, called a heavy chain (H chain) and a light chain (L chain).

Further, the H chain is constituted by regions of an H chain variable region (also referred to as VH) and an H chain constant region (also referred to as CH) from its N-terminal, and the L chain is constituted by regions of an L chain variable region (also referred to as VL) and an L chain constant region (also referred to as CL) from its N-terminal. Regarding CH, α, δ, ε, γ, and μ chains are known for each subclasses. Furthermore, CH is constituted with the respective domains including a CH1 domain, a hinge domain, a CH2 domain, and a CH3 domain from the N-terminal. The CH2 domain and the CH3 domain are collectively called an Fc region or simply Fc. Regarding CL, a Cλ chain and a Cκ chain are known.

The CH 1 domain, hinge domain, CH2 domain, CH3 domain, and Fc region in the present invention can be identified by the number of amino acid residues from the N-terminal according to the EU index [Kabat et al., Sequences of Proteins of Immunological Interest, US Dept. Health and Human Services (1991)]. Specifically, CH1 is identified by the amino acid sequence from positions 118 to 215 in the EU index, the hinge is identified by the amino acid sequence from positions 216 to 230 in the EU index, CH2 is identified by the amino acid sequence from positions 231 to 340 in the EU index, and CH3 is identified by the amino acid sequence from positions 341 to 447 in the EU index, respectively.

A chimeric antibody refers to an antibody consisting of the heavy chain variable region (VH) and the light chain variable region (VL) of a non-human animal antibody and the heavy chain constant region (CH) and the light chain constant region (CL) of a human antibody. For the variable region, any type of animals such as a mouse, rat, hamster, rabbit or the like can be used without limitation, as long as a hybridoma can be prepared therefrom.

A human chimeric antibody can be produced by obtaining cDNAs encoding VH and VL of a non-human animal antibody, inserting them into an expression vector having genes encoding CH and CL of human antibody to construct a human chimeric antibody expression vector, and then introducing the vector into an animal cell to express the antibody. As the CH of the human chimeric antibody, any CH can be used, as long as it belongs to human immunoglobulin (hereinafter, abbreviated to hIg), and those belonging to the hIgG class are preferred. The CL of the human chimeric antibody may be any one of Cκ and Cλ.

A humanized antibody is an antibody in which complementarity-determining regions (hereinafter, abbreviated to CDRs) of VH and VL of a non-human animal antibody are grafted into appropriate positions of VH and VL of a human antibody. The region other than CDRs of VH and VL is referred to as a framework region (hereinafter referred to FR). The human CDR-grafted antibody can be produced by constructing cDNA encoding V regions in which CDRs of VH and VL of a non-human animal antibody are grafted to the framework of VH and VL of any human antibody, inserting each of the cDNAs into an expression vector having DNAs encoding the CH and CL of a human antibody to construct a humanized antibody expression vector, and introducing the expression vector into an animal cell to express the human CDR-grafted antibody. The FR amino acid sequence of VH and VL of human antibody is not particularly limited, as long as it is an amino acid sequence derived from a human antibody.

The CH of the humanized antibody is not particularly limited, as long as it belongs to hIg, and those belonging to the hIgG class are preferred. The CL of the humanized antibody may be any one of Cκ and Cλ.

A human antibody originally means an antibody naturally existing in the human body, but it also includes antibodies obtained from a human antibody phage library and a human antibody-producing transgenic animal, which are prepared based on recent advances in genetic engineering, cell engineering and developmental engineering techniques.

The human antibody can be obtained by immunizing a mouse having human immunoglobulin genes (Tomizuka K. et. al., Proc Natl Acad Sci USA. 97, 722-7, 2000) with a desired antigen. In addition, by selecting a human antibody having a desired binding activity using a phage display library which is formed by antibody gene amplification from human B cells, it is possible to obtain human antibodies without performing immunization (Winter G. et. al., Annu Rev Immunol. 12:433-55.1994). Moreover, by immortalizing human B cells using an EB virus to prepare human antibody-producing cells having a desired binding activity, it is possible to obtain human antibodies (Rosen A. et. al., Nature 267, 52-54.1977).

The antibody existing in the human body can be purified in the following manner, for example; lymphocytes isolated from the human peripheral blood are immortalized by infection with the EB virus or the like, followed by cloning, whereby lymphocytes producing the antibody can be cultured and the antibody can be purified from the culture.

The human antibody phage library is a library of phages which are caused to express antibody fragments such as Fab and scFv on the surface thereof by insertion of antibody genes prepared from the human B cells into the gene of the phage. From this library, it is possible to recover phages which express antibody fragments having a desired antigen binding activity, by using binding activity with respect to an antigen-immobilized substrate as an index. The antibody fragments can be also converted into a human antibody molecule consisting of two complete H chains and two complete L chains by genetic engineering technique.

The human antibody-producing transgenic animal refers to an animal obtained by integration of the human antibody gene into chromosomes of a host animal. Specifically, the human antibody gene is introduced to mouse ES cells, the ES cells are grafted to the early embryo of another mouse, and then the embryo is developed, whereby the human antibody-producing transgenic animal can be prepared. As a method of preparing human antibodies from the human antibody-producing transgenic animal, a human antibody-producing hybridoma is obtained by a normal hybridoma preparation method which is implemented using a mammal other than a human being, followed by culture, whereby human antibodies can be produced and accumulated in the culture.

The antibody fragment used in the therapeutic method of the present invention includes fragments of the respective antibodies. The type of antibody fragment is not particularly limited, and examples thereof may include Fab, Fab′, F(ab′)₂, scFv, diabody, dsFv, a peptide including CDR, or the like.

Fab is an antibody fragment having a molecular weight of about 50,000 and having antigen binding activity, among fragments obtained by treating IgG antibody with papain (protease). Fab can be prepared by treating an antibody with papain, or by inserting DNA encoding Fab of the antibody into an expression vector and introducing this vector into prokaryote or eukaryote for expression.

F(ab′)₂ is an antibody fragment having a molecular weight of about 100,000 and having antigen binding activity, among fragments obtained by treating IgG antibody with pepsin (protease). F(ab′)₂ can be prepared by treating an antibody with pepsin, or through a thioether bond or disulfide bond of Fab′ (described below).

F(ab′) is an antibody fragment of a molecular weight of about 50,000 and having antigen binding activity, in which the disulfide bond of the hinge region of the above F (ab′)₂ is cleaved. F(ab′) can be prepared by treating F(ab′)₂ of an antibody with dithiothreitol, or by inserting DNA encoding Fab′ of the antibody into an expression vector and introducing this vector into prokaryote or eukaryote for expression.

scFV is an antibody fragment having antigen binding activity with a single VH and a single VL which are linked using a suitable peptide linker. scFv can be prepared by obtaining cDNAs encoding VH and VL of an antibody, constructing DNA encoding scFv, inserting this DNA into an expression vector, and introducing this expression vector into prokaryote or eukaryote for expression.

Diabody is an antibody fragment as a dimer formed of scFVs showing the same or different antigen binding specificity, and this antibody fragment has a divalent antigen binding activity with respect to the same antigen or has divalent antigen binding activity with respect to two different types of antigens. Diabody can be prepared by obtaining cDNAs encoding VH and VL of an antibody, constructing DNA encoding diabody, inserting this DNA into an expression vector, and introducing this expression vector into prokaryote or eukaryote for expression.

dsFv is an antibody fragment, in which 1 amino acid residue in each of VH and VL is substituted with a cystine residue, and the polypeptides are linked through a disulfide bond between these cysteine residues. dsFv can be prepared by obtaining cDNAs encoding VH and VL of an antibody, constructing DNA encoding dsFv, inserting this DNA into an expression vector, and introducing this expression vector into prokaryote or eukaryote for expression.

The peptide including CDR is a peptide including at least one or more regions of CDR of VH or VL. The peptide including CDR of an antibody can be prepared by constructing DNA encoding CDR of VH and VL of the antibody, inserting this DNA into an expression vector, and introducing this expression vector into prokaryote or eukaryote for expression. The peptide including CDR can be also prepared by chemical synthesis method such as an Fmoc method (fluorenyl methyloxycarbonyl method) or a tBoc method (t-butoxycarbonyl method).

In the present invention, the effector activity refers to an activity induced via the Fc region of an antibody. As the effector activity, antibody-dependent cellular cytotoxicity activity (ADCC activity), complement-dependent cytotoxicity activity (CDC activity), and antibody-dependent phagocytosis (ADP activity) caused by phagocytes such as granulocytes, macrophages, dendritic cells or the like are known.

The ADCC activity refers to an activity in which an antibody bound to an antigen on a target cell binds to an Fc receptor of an immunocyte via the Fc region of the antibody, thereby activating the immunocyte (a natural killer cell or the like) and damaging the target cell.

The Fc receptor (hereinafter, referred to as FcR in some cases) refers to a receptor binding to the Fc region of an antibody, and induces various types of effector activity due to the binding of an antibody.

FcR corresponds to antibody subclasses, and IgG, IgB, IgA and IgM specifically bind to FcγR, FcεR, FcαR and FcμR respectively. FcγR has subtypes including FcγRI (CD64), FcγRII (CD32) and FcγRIII (CD16), and each subtype has isoforms including FcγRIA, FcγRIB, FcγRIC, FcγRIIA, FcγRIIB, FcγRIIC, FcγRIIIA, and FcγRIIB. These different types of FcγR exist on different cells [Annu. Rev. Immunol. 9:457-492(1991)].

In human beings, FcγRIIIB is specifically expressed in neutrophils, and FcγRIIIA is expressed in monocytes, natural killer cells (NK cells), and a portion of T cells. The antibody binding via FcγRIIIA induces NK cell-dependent ADCC activity.

The CDC activity refers to an activity in which an antibody bound to an antigen on a target cell activates a series of cascades (complement activation pathways) consisting of a group of complement-related proteins in the blood, thereby damaging the target cell. By the protein fragments generated due to the complement activation, it is possible to induce migration and activation of immunocytes. In the cascades of CDC activity, when C1q having a binding domain for the Fc region of an antibody binds to the Fc region, and C1r and C1s as two serine proteases bind thereto, a C1 complex is formed, whereby the cascade of CDC activity begins.

In the present invention, phagocytosis includes any one of phagocytosis mediated by C3 receptor on the phagocyte induced by deposition of the complement C3b or C3d on the bacterial surface which is generated in an intermediate reaction during complement activation cascade initiated by binding of an antibody to a molecule on the bacterial surface and binding of the complement factor C1q to the antibody, and phagocytosis induced by binding of the Fab region of an antibody to a molecule on the bacterial surface and binding of the Fc region of the antibody to the Fc receptor on phagocyte. As such, the activity of phagocytizing bacteria that are opsonized with the complement factor or the antibody is called opsonophagocytosis.

In the present invention, the phagocyte may be any cell, as long as it has the phagocytosis, and specific examples thereof may include granulocytes, macrophages, and dendritic cells. Examples of the granulocytes may include neutrophils, basophils, and eosinophils. More preferably, examples of phagocyte may include neutrophils, macrophages and dendritic cells. Phagocytes show different levels of phagocytosis depending on activation state and phagocytes can be in any activation state, as long as they are able to cause phagocytosis.

In the present invention, the C3 receptor and the Fc receptor expressed on phagocytes may be Mac-1 and FcγRIIIB, respectively.

As a method for controlling the effector activity, a method of controlling the amount of the fucose (also called core fucose) which is bound to N-acetylglucosamine (GlcNAc) through α-1,6 bond in a reducing end of a complex-type N-linked sugar chain which is bound to asparagine (Asn) at position 297 according to EU index (Kabat et at, Sequence of Proteins of Immunological Interests, 5th edition, 1991) of an Fc region of an antibody (WO 2005/035586, WO 2002/31140, WO 00/61739), a method of controlling the activity by substituting amino acid residues of Fc region of the antibody, or the like is known.

Therefore, the effector activity of the antibody can be increased or decreased by controlling the content of core fucose of the complex-type N-linked sugar chain bound to the Fc region of the antibody binding to a molecule on the bacterial surface, in which the antibody is modified by substituting at least one amino acid residue so as to show more enhanced CDC than the antibody before substitution of the amino acid residue. The method for decreasing the content of fucose to be bound to the complex-type N-linked sugar chain bound to the Fc region of the antibody is to obtain an antibody having no fucose binding thereto by expressing an antibody using CHO cell from which α1,6-fucosyltransferase gene (fucosyltransferase-8, FUT8) is deleted.

The antibody having no fucose binding thereto has high ADCC activity and high phagocytosis. On the other hand, the method for increasing the content of fucose to be bound to the complex-type N-linked sugar chain bound to the Fc region of the antibody is to obtain the antibody having fucose binding thereto by expressing the antibody using a host cell in which α1,6-fucosyltransferase gene is introduced. The antibody having fucose binding thereto has lower ADCC activity and phagocytosis than the antibody having no fucose binding thereto.

Therefore, the antibody of the present invention may be a composition that is composed of antibody molecules having the same or the different sugar chain(s). The antibody composition is a composition composed of antibody molecules having the Fc region, in which the complex-type N-glycoside linked sugar chain is bound to Asn at position 297 from the N-terminal of the Fc region of the antibody molecule.

In the present specification, a ratio of the sugar chain having no core fucose refers to a ratio of the number of the complex-type N-glycoside linked sugar chain having no core fucose to the total number of the complex-type N-glycoside linked sugar chain binding to Fc of the antibody molecule included in the composition.

The ratio of the sugar chain having no core fucose may be any ratio in the antibody composition, as long as ADCC activity and phagocytosis of the antibody are increased. It may be preferably 20% or more, more preferably 51%-100%, much more preferably 80%-100%, particularly preferably 90%, 91%, 92%, 93%, 94%, 95%, 96%, 97%, 98%, 99%, and most preferably 100%. In the present invention, the antibody composition having no core fucose is defined as Potelligent (PT), non-fucosylated antibody or Fuc(−)IgG.

The antibody composition having 50% of the ratio of the sugar chain having no core fucose encompasses both of an antibody composition including 100% of molecules having no fucose at one sugar chain of the N-glycoside linked sugar chains bound to two H chains of the antibody molecule, and an antibody composition including 50% of molecules having no fucose at both sugar chains of the N-glycoside linked sugar chains bound to two H chains of the antibody molecule and 50% of molecules having fucose at both sugar chains of the N-glycoside linked sugar chains bound to two H chains of the antibody molecule.

In the present invention, the antibody composition having 20% or more of the ratio of the sugar chain having no core fucose shows the increased binding activity for FcγRIIIb on phagocyte, thereby increasing phagocyte-dependent phagocytosis.

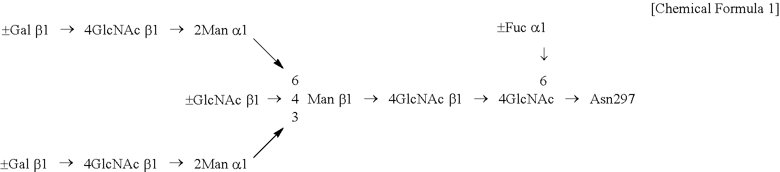

In the present invention, the sugar chain having no fucose may have any structure of the sugar chain at the non-reducing end, as long as fucose does not bind to N-acetylglucosamine at the reducing end in the above Chemical Formula.

Further, ADCC activity or CDC activity can be increased or decreased by substituting the amino acid residues of the Fc region of the antibody. The binding activity for FcγR can be increased or decreased by substituting the amino acid residues of the Fc region, thereby controlling ADCC activity. The binding activity of complement can be increased or decreased by substituting the amino acid residues of the Fc region, thereby controlling CDC activity.

For example, CDC activity of the antibody can be increased by using the amino acid sequence of the Fc region, which is described in the specification of US Patent Application Publication Nos. 2007/0148165 and 2012/0010387. Also, ADCC activity or CDC activity can be increased or decreased by carrying out substitution of the amino acid residues, which is described in the specification of U.S. Pat. Nos. 6,737,056, 7,297,775, and 7,317,091 and WO 2005/070963.

In the present invention, the increased deposition of complement C3b on the bacterial surface means that the binding amount of C3b on the bacterial surface caused by binding of the antibody to the molecule on the bacterial surface is increased by the modified antibody of the present invention, compared to the binding amount of C3b caused by the antibody before substitution of amino acid residues.

In the present invention, the increased phagocytosis by phagocyte means that phagocytosis by phagocyte caused by binding of the antibody to the molecule on the bacterial surface is increased by the modified antibody of the present invention, compared to phagocytosis caused by the antibody before substitution of amino acid residues.

In the present invention, the reduced bacterial proliferation means that cell proliferation of bacteria ingested by phagocytes through the above described phagocytosis is reduced or inhibited, compared to the bacteria cell proliferation before ingestion by phagocyte, and the bacteria ingested by phagocytes are damaged to be killed (sterilized) or destroyed.

In the present invention, the modified antibody is an antibody in which at least one amino acid residue included in the Fc region of a natural antibody molecule is substituted with other amino acid residue, and includes modified antibodies having higher CDC than CDC induced by human IgG1 antibody and human IgG3 antibody including VH and VL amino acid sequences identical to those of modified antibody. The position and type of amino acid residue to be substituted may be any position and type of amino acid residue, as long as the substitution is performed to obtain higher CDC than CDC induced by human IgG1 antibody and human IgG3 antibody including VH and VL amino acid sequences identical to those of modified antibody. Substitution of at least one amino acid residue included in CH2 domain is preferred.

In the present invention, the modified antibody includes human IgG1 antibody binding to a molecule on the bacterial surface, in which at least one amino acid residue in the Fc region is substituted with other amino acid residue, and it has higher CDC activity than the CDC activity induced by human IgG1 antibody before modification and human IgG3 antibody including VH and VL amino acid sequences identical to those of human IgG1 antibody.

Specific substitution of amino acid residue for increasing CDC activity may include substitution of at least one amino acid residue selected from K326A, S267E, H268F, S324T, K274Q, N276K, Y296F, Y300F, K326W, K326Y, E333A, E333S, A339T, D356E, L358M, N384S, K392N, T394F, T394Y, V397M and V422I.

Preferably, the substitution of amino acid residue for increasing CDC activity may include substitution of at least one amino acid residue selected from N276K, A339T, T394F and T394Y, substitution of amino acid residues of N276K and A339T, substitution of amino acid residues of K274Q, N276K, Y296F, Y300F, A339T, D356E, L358M, N384S, K392N, V397M and V422I, and substitution of amino acid residues of K274Q, N276K, Y296F, Y300F, A339T, D356E, L358M, N384S, V397M and V422I (an amino acid residue is represented by 1 letter, the letter before a number indicates the amino acid residue before substitution, and the letter after the number indicates the amino acid residue after substitution. In addition, all numbers of the amino acid residues are represented based on the EU index).

Specific examples of the modified antibody used in the therapeutic method and therapeutic agent of the present invention may include modified antibodies including substitution of at least one amino acid residue selected from K326A, S267E, H268F, S324T, K274Q, N276K, Y296F, Y300F, K326W, K326Y, E333A, E333S, A339T, D356E, L358M, N384S, K392N, T394F, T394Y, V397M and V422I. Preferred examples thereof may include a modified antibody including substitution of at least one amino acid residue selected from N276K, A339T, T394F and T394Y, a modified antibody including substitution of amino acid residues of N276K and A339T, a modified antibody including substitution of amino acid residues of K274Q, N276K, Y296F, Y300F, A339T, D356E, L358M, N384S, K392N, V397M and V422I, and a modified antibody including substitution of amino acid residues of K274Q, N276K, Y296F, Y300F, A339T, D356E, L358M, N384S, V397M and V422I as the amino acid sequence of the Fc region of human IgG1 antibody that is described in the specification of US Patent Application Publication NOs. 2007/0148165 and 2012/0010387.

Further, the modified antibody used in the therapeutic method and therapeutic agent of the present invention is preferably a modified antibody and a modified antibody composition, in which the modified antibody is the above described modified antibody and the ratio of the sugar chain having no core fucose in the antibody is 20% or more.

In the present invention, in some cases, the modified antibody including substitution of amino acid residues of K274Q, N276K, Y296F, Y300F, A339T, D356E, L358M, N384S, K392N, V397M and V422I is also defined as Complegent (registered trademark), the modified antibody including substitution of amino acid residues of K274Q, N276K, Y296F, Y300F, A339T, D356E, L358M, N384S, K392N, V397M and V422I and having no core fucose bound thereto is also defined as AccretaMab (registered trademark), the modified antibody including substitution of amino acid residues of K274Q, N276K, Y296F, Y300F, A339T, D356E, L358M, N384S, V397M and V422I is also defined as Neocomplegent, and the modified antibody including substitution of amino acid residues of K274Q, N276K, Y296F, Y300F, A339T, D356E, L358M, N384S, V397M and V422I and having no core fucose bound thereto is also defined as NcoAccretaMab.

In the present invention, the bacteria may be bacteria belonging to any genus and species, as long as they are effective in the therapeutic method of the present invention. Specific examples thereof may include Gram-positive and Gram-negative bacteria.

Examples of the Gram-positive bacteria may include Staphylococcus, Streptococcus, Pneumococcus, Mycoplasma, Listeria, Corynebacterium dyphtheriae, Bacillus anthracis, Clostridium botulinum, Clostridium tetani, Clostridium perfringens, Mycobacterium, Mycobacterium tuberculosis, Mycobacterium leprae or the like.

Examples of the Gram-negative bacteria may include Neisseria gonorrhoeae, Neisseria meningitidis, Shigella, Escherichia coli, Salmonella, S. typhi, Haemophilus influenzae, Klebsiella pneumoniae, Bordetella pertussis, Vibrio cholerae, Campylobacter, Pseudomonas aeruginosa, Legionella pneuophila, Bacteroides, Spirochete: syphilis, leptospira, borrelia (Lyme disease), Chlamydia: trachoma, psittaci, non-gonococcal urethritis, Rickettsia: tsutsugamushi or the like.

The target of the therapeutic method of the present invention is more preferably Gram-positive bacteria having a thick proteoglycan cell membrane, such as Staphylococcus aureus, Streptococcus pneumonia, Bacillus antracis, Clostridium botulinum, Clostridium tetani, Clostridium perfringens, Mycoplasma pneumoniae or the like.

The bacteria of the present invention also include variants of the above described bacteria being resistant to one or more drugs.

Specific examples thereof may include bacteria being resistant to 3-lactam antibiotics such as meticillin, amoxicillin or the like, or resistant to macrolide antibiotics such as erythromycin, vancomycin or the like. More specific examples thereof may include meticillin resistant Staphylococcus aureus (abbreviated to MRSA), vancomycin intermediate Staphylococcus aureus (abbreviated to VISA), vancomycin resistant Staphylococcus aureus (abbreviated to VRSA) or the like.

In the present invention, the molecule on the bacterial surface may be any of proteins, glycoproteins, glycolipids, and lipopolysaccharides that are expressed by the above described bacteria. Specific examples thereof may include capsular polysaccharide (CP), ganglioside, pncumococcal surface protein (Psp), and lipopolysaccharide (LPS).

More specific examples thereof may include Staphylococcus aureus capsular polysaccharide 5 (SACP5), SACP8, pneumonial surface protein A (PspA), pneumococcal surface protein C (PspC), ganglioside GD3 or the like.

The therapeutic method of the present invention also includes a combination therapy of the antibody with other therapeutic agents.

The combination therapy of the present invention may be a combination therapy of antibiotics with an antibody selected from the antibody binding to a molecule on the bacterial surface, which is modified by substituting at least one amino acid residue with other amino acid so as to show more enhanced CDC than the antibody before substitution of the amino acid residue, the antibody binding to a molecule on the bacterial surface, in which the ratio of the complex type N-linked sugar chain having no α1,6-fucose bound thereto is 20% or more, and the antibody binding to a molecule on the bacterial surface, in which the antibody is modified by substituting at least one amino acid residue with other amino acid so as to show more enhanced CDC than the antibody before substitution of the amino acid residue and the ratio of the complex type N-linked sugar chain having no α1,6-fucose bound thereto is 20% or more.

The therapeutic agent of the present invention may be any therapeutic agent, as long as it includes the antibody having the above described activity as an active ingredient. Typically, it is preferable that the therapeutic agent is provided in the form of a pharmaceutical preparation which is produced by mixing with one or more pharmaceutically acceptable carriers according to any method known in the pharmaceutics.

Preferably, an aseptic solution prepared by dissolving the antibody in an aqueous carrier such as an aqueous solution (e.g., water, saline, glycine, glucose, human albumin, or the like) is used. Further, a pharmaceutically acceptable additive such as a buffer or an isotonic agent (e.g., sodium acetate, sodium chloride, sodium lactate, potassium chloride, sodium citrate or the like) may be added to the preparation solution to approach physiological conditions. Further, it may be freeze-dried and then stored. If necessary, it may be dissolved in an appropriate solvent when using.

It is preferable that the therapeutic agent of the present invention is administered via the route being most effective for the treatment. Examples thereof may include oral administration and parenteral administration such as intraoral, intratracheal, intrarectal, subcutaneous, intramuscular, intrathecal and intravenous administrations. Intrathecal or intravenous administration is preferred.

Examples of the preparation suitable for oral administration may include emulsions, syrups, capsules, tablets, powders, granules or the like. Liquid preparations such as emulsions and syrups can be produced using, as additives, water, sugars such as sucrose, sorbitol, fructose or the like, glycols such as polyethylene glycol, propylene glycol or the like, oils such as sesame oil, olive oil, soybean oil or the like, antiseptics such as p-hydroxybenzoate esters or the like, flavors such as strawberry flavor, peppermint, or the like. Capsules, tablets, powders, granules or the like can be produced using, as additives, excipients such as lactose, glucose, sucrose, mannitol or the like, disintegrating agents such as starch, sodium alginate or the like, lubricants such as magnesium stearate, talc or the like, binders such as polyvinyl alcohol, hydroxypropylcellulose, gelatin or the like, surfactants such as fatty acid ester or the like, plasticizers such as glycerin or the like.

Examples of the preparation suitable for parenteral administration may include injections, suppositories, sprays or the like. Injections can be prepared using a carrier such as a salt solution, a glucose solution and a mixture of both thereof. Suppositories can be prepared using a carrier such as cacao butter, hydrogenated fat, carboxylic acid or the like. Sprays can be prepared using the antibody as it is, or using it together with a carrier which does not stimulate the buccal or airway mucous membrane of the recipient and can facilitate absorption of the antibody by dispersing it as fine particles or the like. Specific examples of the carrier may include lactose, glycerol or the like. It is possible to produce preparations such as aerosols and dry powders, depending on the properties of the antibody and the carriers used. In addition, the components exemplified as additives for oral preparations can also be added to the parenteral preparations.

The administration dose or frequency of the therapeutic agent of the present invention will vary depending on the desired therapeutic effect, the administration route, the period of treatment, age, body weights or the like. The administration dose for an adult person is typically 1 μg/kg-10 mg/kg per day.

The present invention also includes a method for increasing deposition of complement C3b on the bacterial surface and phagocytosis by phagocytes using the modified antibody binding to a molecule on the bacterial surface, which includes substitution of at least one amino acid residue and shows more enhanced complement-dependent cytotoxicity (CDC).

Hereinafter, the present invention will be described in detail with reference to Examples. However, the present invention is not limited to these Examples.

In the following Examples, IgG antibody having core fucose bound to the complex type N-linked sugar chain binding to the Fc of the antibody is represented by conventional (hereinafter, abbreviated to con), the antibody having no core fucose bound thereto is represented by Potelligent (registered trademark) (hereinafter, abbreviated to PT), the antibody having enhanced CDC by substitution of amino acid residues of the Fc region is represented by Completent (hereinafter, abbreviated to CM) or Neocomplegent (hereinafter, abbreviated to NCM), and the antibody having no core fucose bound to the complex type N-linked sugar chain binding to the Fc of the antibody and also having enhanced CDC by substitution of amino acid residues of the Fc region is represented by AccretaMab (hereinafter, abbreviated to Ac) or NeoAccretaMab (hereinafter, abbreviated to NAc).

### EXAMPLES

### Example 1

### Evaluation of Binding Activity of Anti-Ganglioside GD3 Antibody for FcγRIII

Fusion proteins were prepared by linking His tags to the amino acid sequences of extracellular regions of human FcγRIIIA and human FcγRIIIB, and used to evaluate the binding activity of anti-ganglioside GD3 antibody KW-2871 by a surface plasmon resonance method (SPR) using Biacore 3000. Human FcγRIIIA was prepared as two gene polymorphisms of 158Val and 158Phe, and human FcγRIIIB was prepared as two polymorphic variants of NA1 and NA2.

The anti-ganglioside GD3 antibody KW-2871 was prepared as fucosylated KW-2871 (con-KW-2871) having core fucose and non-fucosylated KW-2871 (PT-KW-2871) having no core fucose, and used as samples. These antibodies were prepared by the method of WO 2011/068136.

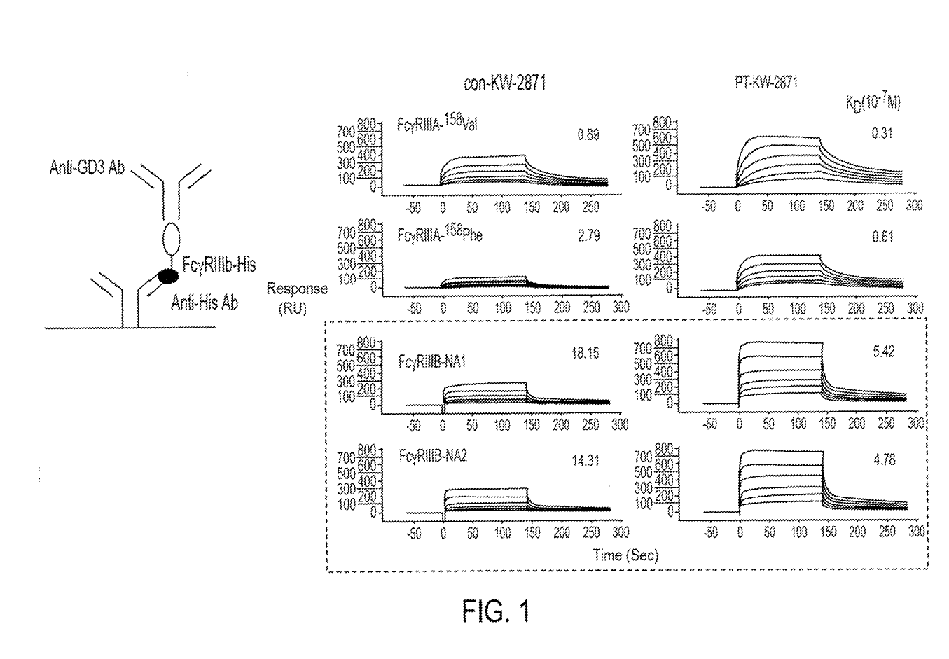

As shown in FIG. 1, the results showed that PT-KW-2871 bound to any of FcγRIII with a stronger affinity than con-KW-2871.

### Example 2

### Evaluation of Anti-Ganglioside GD3 Antibody-Mediated Phagocytosis

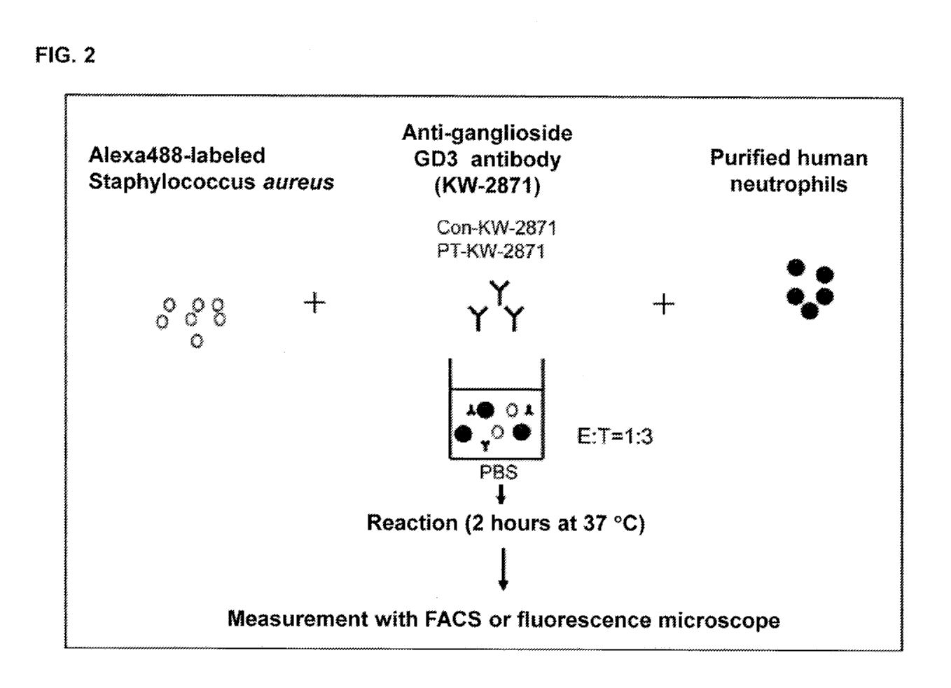

The opsonophagocytosis mediated by con-KW-2871 and PT-KW-2871 was compared using polymorphonuclear neutrophils (hereinafter, abbreviated to PMN) and Staphylococcus aureus strain. As represented in FIG. 2, Staphylococcus aureus labeled with fluorescent dye Alexa 488 were reacted with PBS (negative control) or serial concentrations of each antibody in the presence of human neutrophils. After 15 min from the start of reaction, cold PBS was added to the reaction solution to stop neutrophil phagocytosis.

The bacterial cells and the neutrophils in the reaction solution were washed twice, and the ratio of neutrophils that ingested bacteria into the cell was measured using flow cytometer and fluorescence microscope. Ethidium bromide was added to the samples to distinguish between ingested bacteria and not-ingested bacteria by neutrophils.

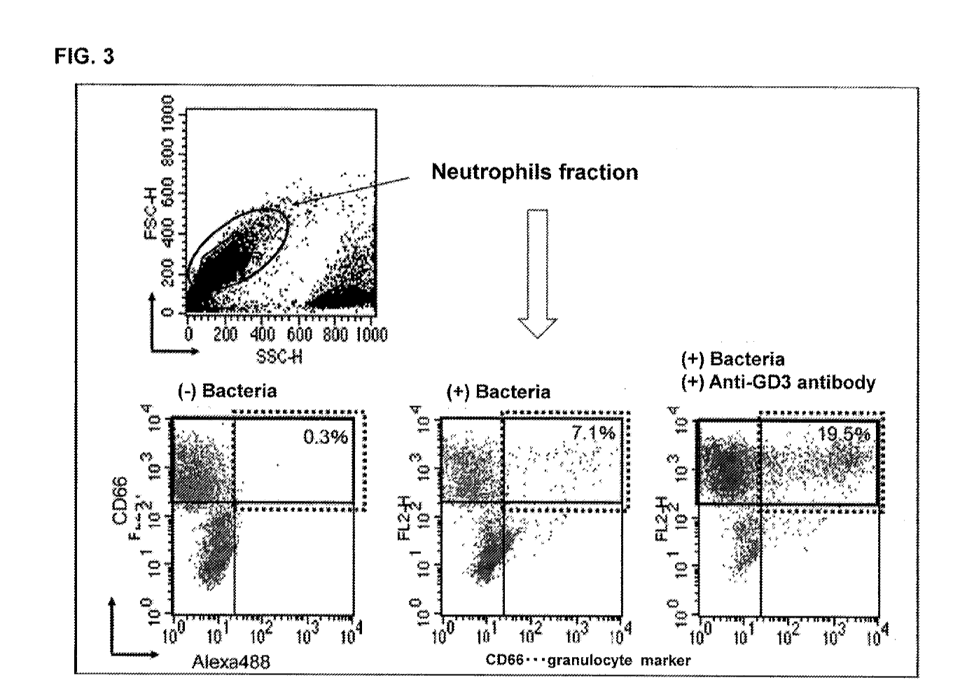

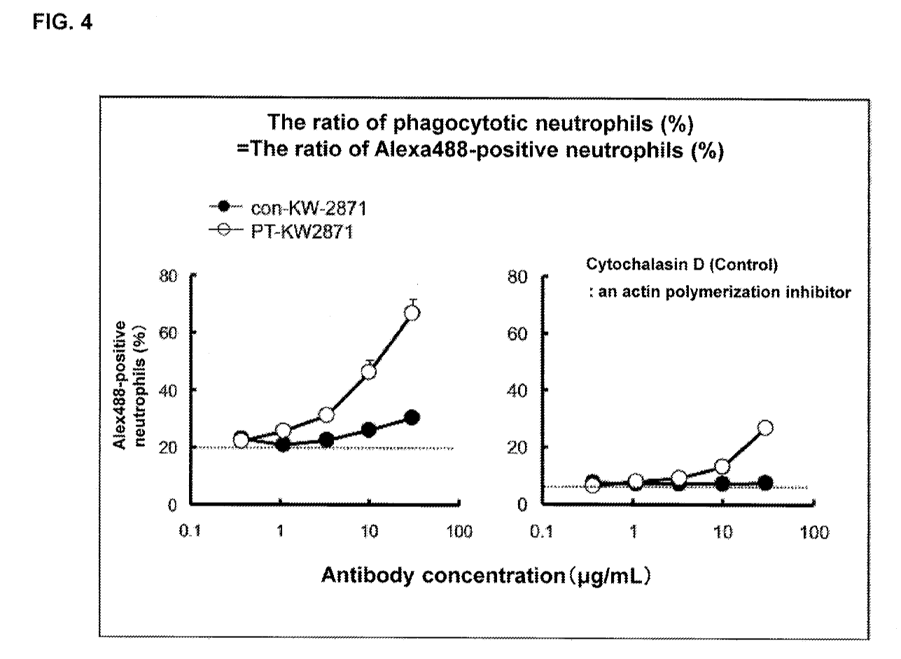

As a result, as represented in FIGS. 3 to 5, the ingestion of Staphylococcus aureus by neutrophils was increased in the presence of PT-KW-2871 than in the presence of con-KW-2871.

### Example 3

### Evaluation of Anti-Ganglioside GD3 Antibody-Mediated Deposition of Complement C3b on Bacterial Surface

Anti-ganglioside GD3 antibodies KW-2871 were used as samples in forms Ac-KW-2871 and CDC-deficient-KW-2871. The Ac-KW-2871 was constructed as an antibody that did not have core fucose bound to the complex type N-linked sugar chain attached to Fc of antibody, and that had enhanced CDC by amino acid residue substitution in the Fc region. The CDC-deficient-KW-2871 antibody lacked CDC. The amino acid sequence of the Fc region of Ac-KW-2871 is represented by SEQ ID NO: 1. The Ac-KW-2871 and CDC-deficient-KW-2871 were constructed by using the amino acid sequences of the variable regions described in the specification of WO2011/068136, using the method described in the specification of US Patent Application Publication 2007/0148165 for the Ac-KW-2871, and the method of U.S. Pat. No. 6,242,195 for the CDC-deficient-KW-2871.

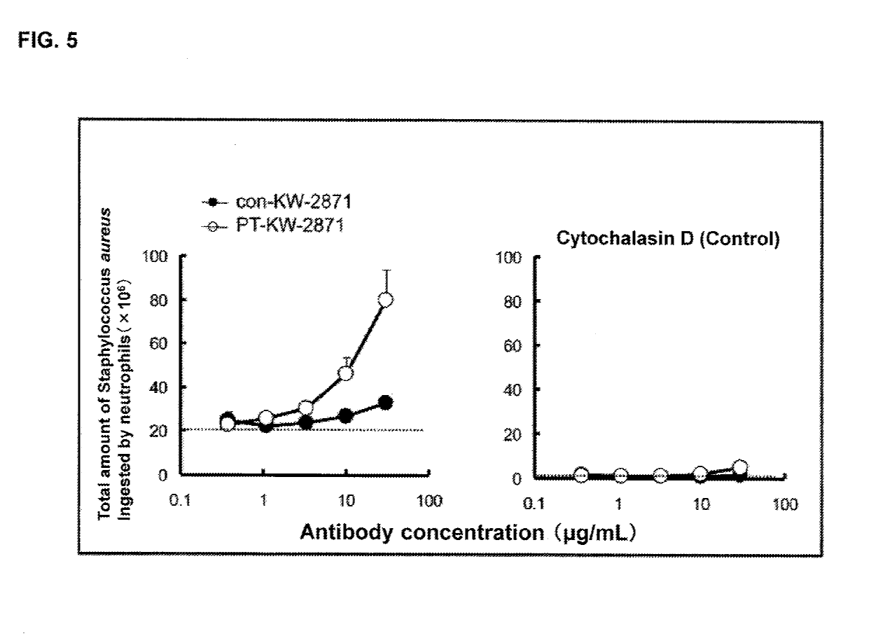

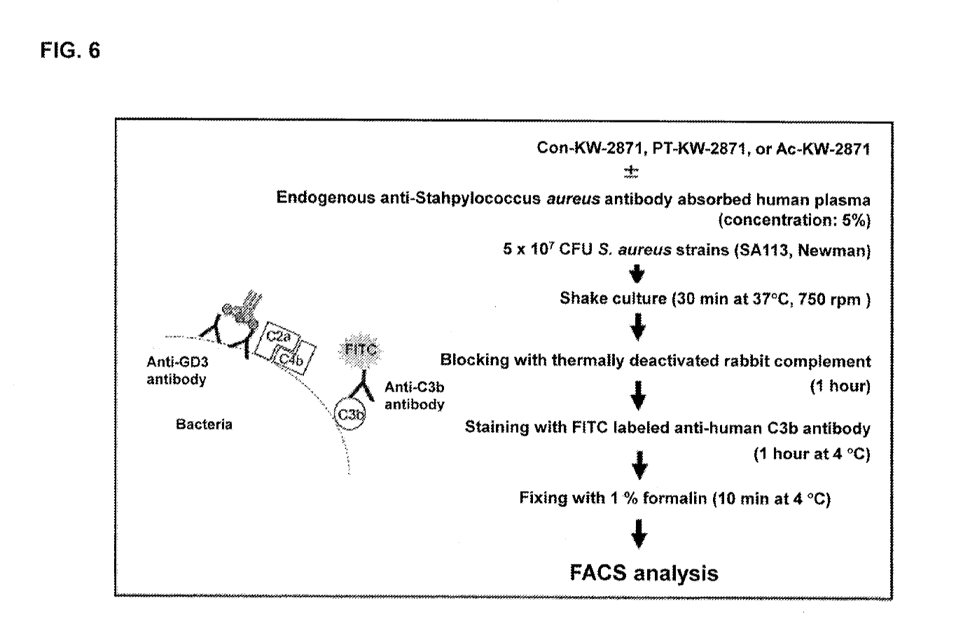

The activity mediated by con-KW-2871, PT-KW-2871, Ac-KW-2871, and CDC-deficient-KW-2871 for the deposition of complement C3b on bacterial surface was assayed with human complement and Staphylococcus aureus strain Newman (ATCC 25905) or SA 13 (ATCC 35556) using the method of FIG. 6. Referring to FIG. 6, the plasma adsorbing the endogenous anti-Staphylococcus aureus antibody was obtained after removing endogenous anti-Staphylococcus aureus immunoglobulins by 1-hour reaction of human-derived plasma with Staphylococcus aureus strain on ice repeated three times. As represented in FIG. 6, phagocyte phagocytizes the C3b-deposited bacteria via C3b receptor such as Mac-1 or CR1. Further, Gram-positive bacteria are not lysed by CDC.

Experiment was conducted in triplicate. Mean fluorescence intensity (MFI) and standard deviation are shown in the graphs. *, p<0.05; two-tailed unpaired t-test. Anti-dinitrophenylhydrazine (hereinafter, referred to as DNP) antibody was used as isotype control.

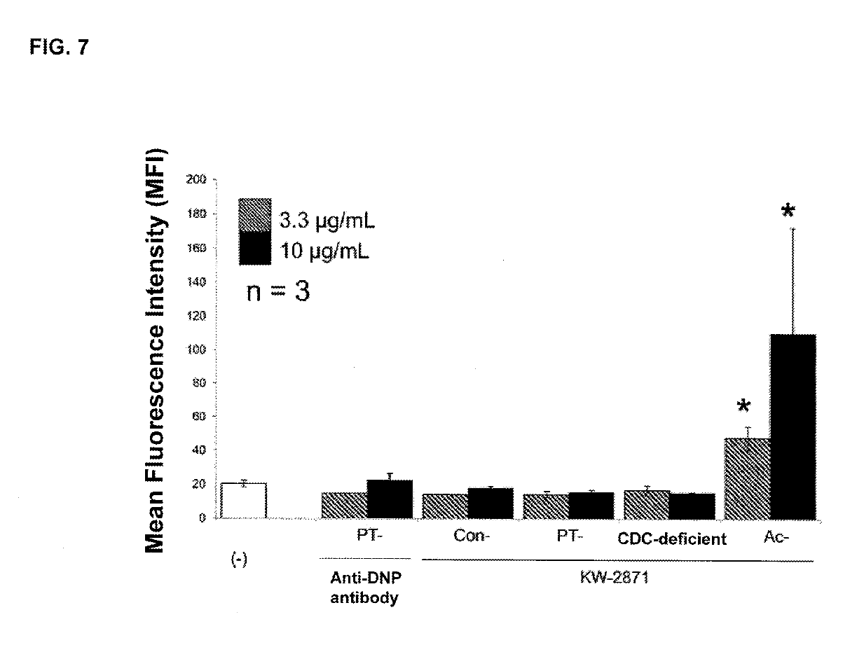

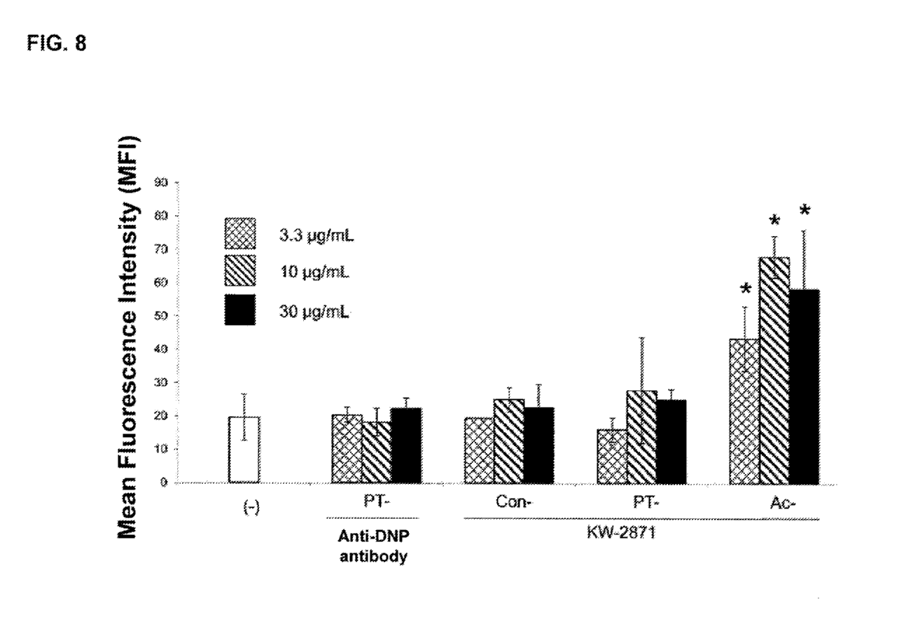

As a result, as represented in FIGS. 7 and 8, only the Ac-KW-2871 antibody among the antibodies used induced complement C3b deposition on Newman and SA 13 surfaces in antibody concentration-dependent manner.

### Example 4

### Evaluation of Anti-Ganglioside GD3 Antibody-Mediated Opsonophagocytosis

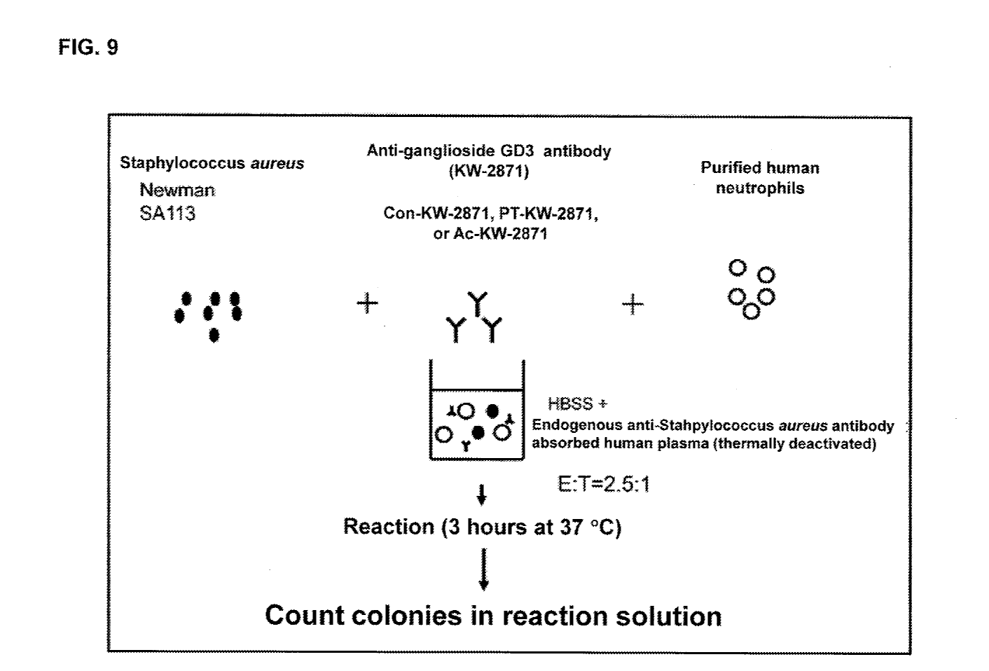

The opsonophagocytosis mediated by con-KW-2871, PT-KW-2871, and Ac-KW-2871 was assayed with human polymorphonuclear neutrophils, as shown in FIG. 9. Agar-grown Newman was reacted with purified human polymorphonuclear neutrophils and each antibody. Human polymorphonuclear neutrophils were taken from two donors, and human polymorphonuclear neutrophils (effector cells) and bacteria (target) were used in a 2.5:1 ratio.

As negative control, samples with addition of buffer instead of human polymorphonuclear neutrophils were used. Experiment was conducted in quadruplicate. Average values and standard deviation of colony-forming unit concentration (CFU/mL) are shown in the graph. The dotted line indicates the CFU concentration at the 0 hr time culture (˜3.1×10⁷ CFU/mL). *, p<0.01, two-tailed unpaired t-test.

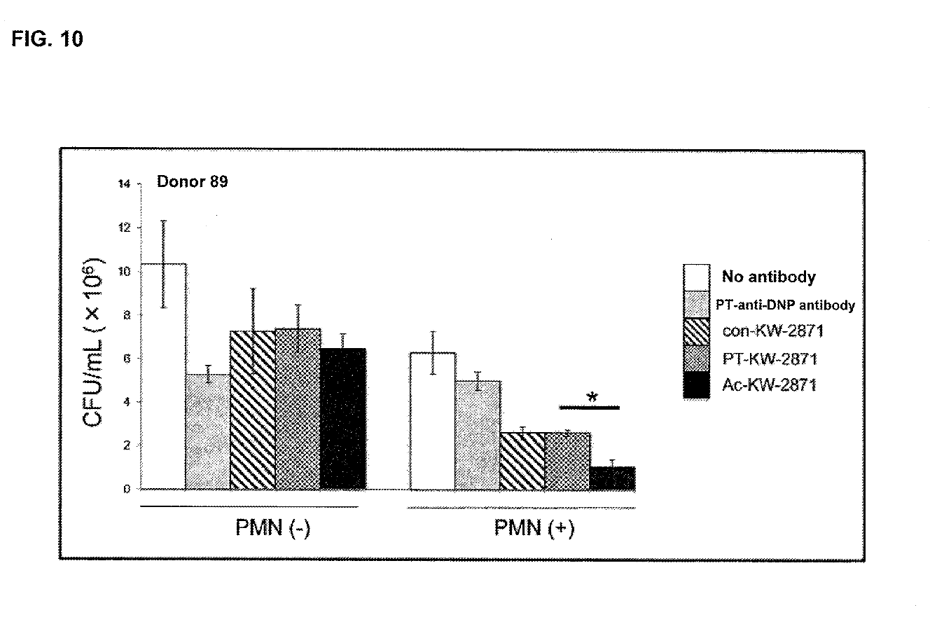

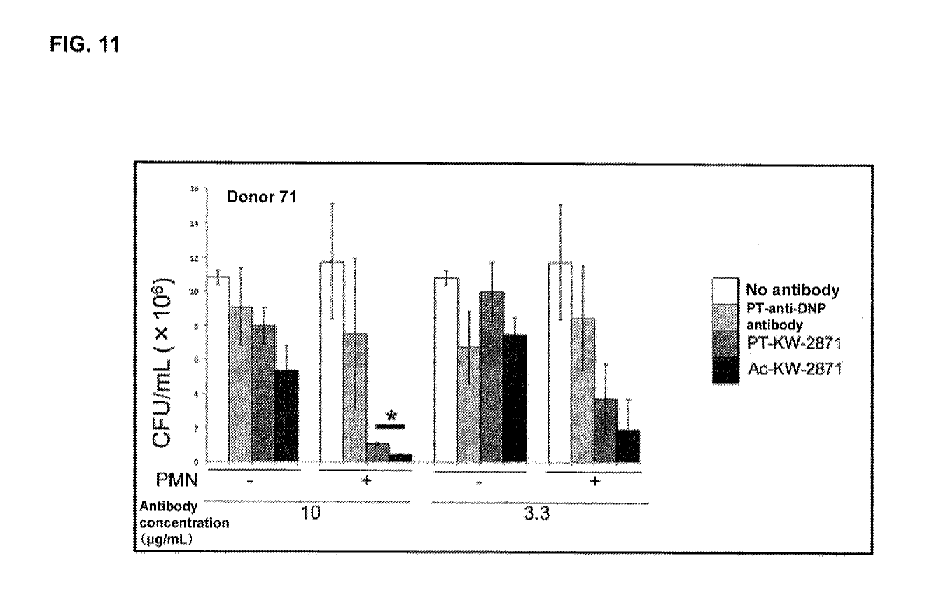

As a result, as represented in FIGS. 10 and 11, Ac-KW-2871-mediated opsonophagocytosis against the Staphylococcus aureus strain was stronger than that of PT-KW-2871. Further, PT-KW-2871-mediated opsonophagocytosis was likely stronger than that of con-KW-2871.

### Example 5

### Evaluation of Anti-CP5 Antibody-Mediated Deposition of Complement C3b on Staphylococcus aureus Strain Surface

An anti-staphylococcus aureus capsular polysaccharide 5 (hereinafter, referred to as SACP5 or CP5) mouse antibodies were established from mice immunized with a purified capsular polysaccharide 5 from Staphylococcus aureus strain Reynolds. The anti-CP5 monoclonal antibody 137G18A was reconstructed to anti-CP5 chimeric antibody 137G18A by using an ordinary method. The nucleotide sequence and the amino acid sequence of VH of the anti-CP5 monoclonal antibody 137G18A are represented by SEQ ID NOS: 3 and 4, respectively. The nucleotide sequence and the amino acid sequence of VL of the anti-CP5 monoclonal antibody 137G18A are represented by SEQ ID NOS: 5 and 6, respectively.

The following antibodies were constructed and used as samples. IgG antibody con-137G18A with core fucose bound to the complex type N-linked sugar chain attached to Fc of anti-CP5 chimeric antibody 137G18A; PT-137G18A antibody without core fucose bound to the complex type N-linked sugar chain attached to Fc; CM-137G18A antibody with enhanced CDC by amino acid residue substitution in the Fc region; and Ac-137G18A antibody without core fucose bound to the complex type N-linked sugar chain attached to Fc and with enhanced CDC by amino acid residue substitution in the Fc region.

CM-137G18A and Ac-137G18A were constructed by using the method described in the specification of US Patent Application Publication 2007/0148165. The amino acid sequence of the Fc region is represented by SEQ ID NO: 1.

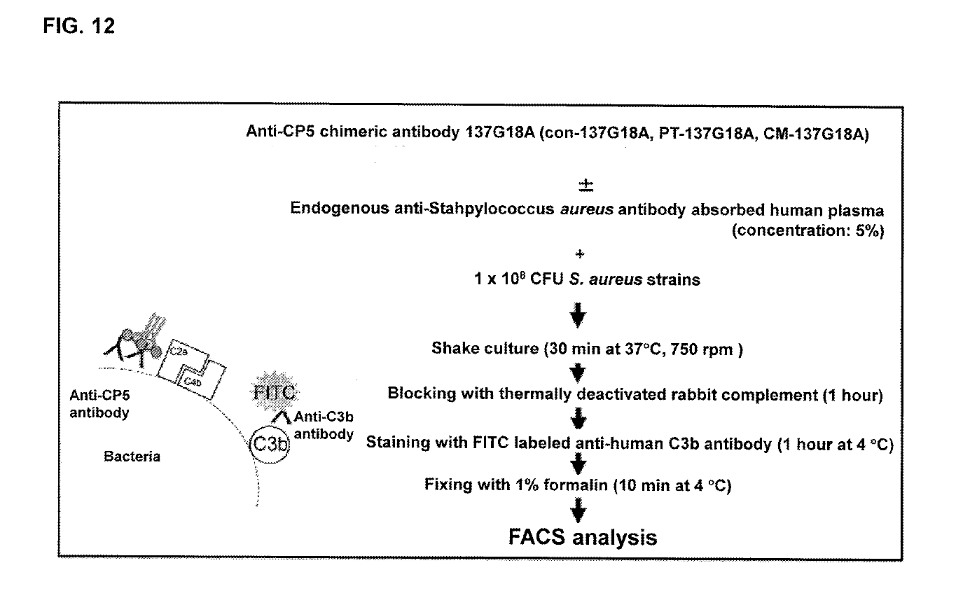

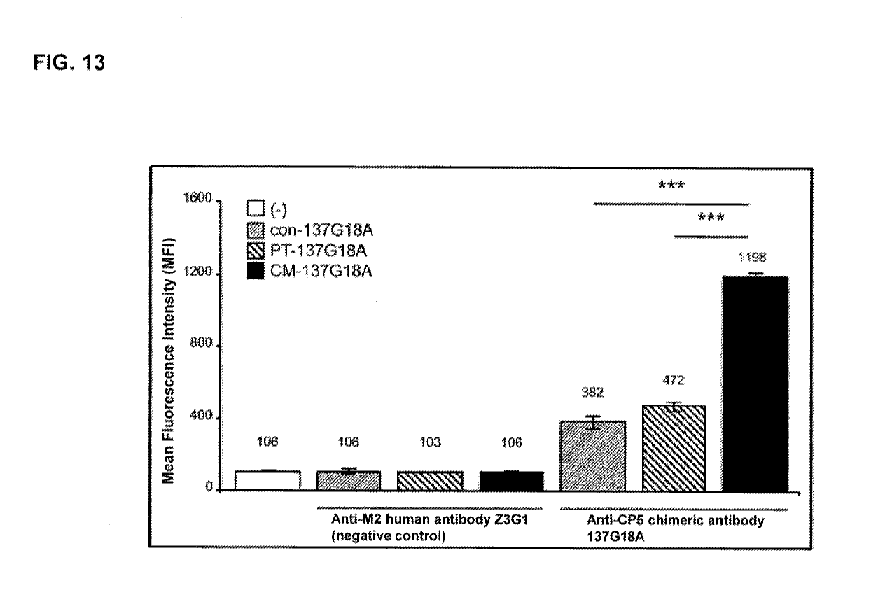

Con-137G18A, PT-137G18A, and CM-137G18A were assayed for complement deposition activity for bacterial surface in the presence of human serum using the method shown in FIG. 12. The results are shown in FIG. 13.

As the experimental method, 5×10⁸ CFU/mL (total amount 10⁸ CFU) Staphylococcus aureus strain Lowenstein were reacted in 2.5% human complement-supplemented RPMI (200 μL) for 15 min in the presence or absence of 2.5 mg/mL (total amount 0.5 mg) of control antibody and anti-CP5 chimeric antibody. C3b deposition indicative of complement deposition on bacteria was then measured by FACS analysis using FITC-labeled anti-C3b antibody. Experiment was conducted in triplicate or sextuplicate. Average values and standard deviation are shown in the graph of FIG. 13. (***, p<0.0005; two-tailed unpaired t-test.)

As a result, as shown in FIG. 13, PT-137G18A had the tendency to show higher C3b deposition activity for bacterial surface than con-137G18A. The CM-137G18A antibody showed the highest C3b deposition activity for bacterial surface among the antibodies used.

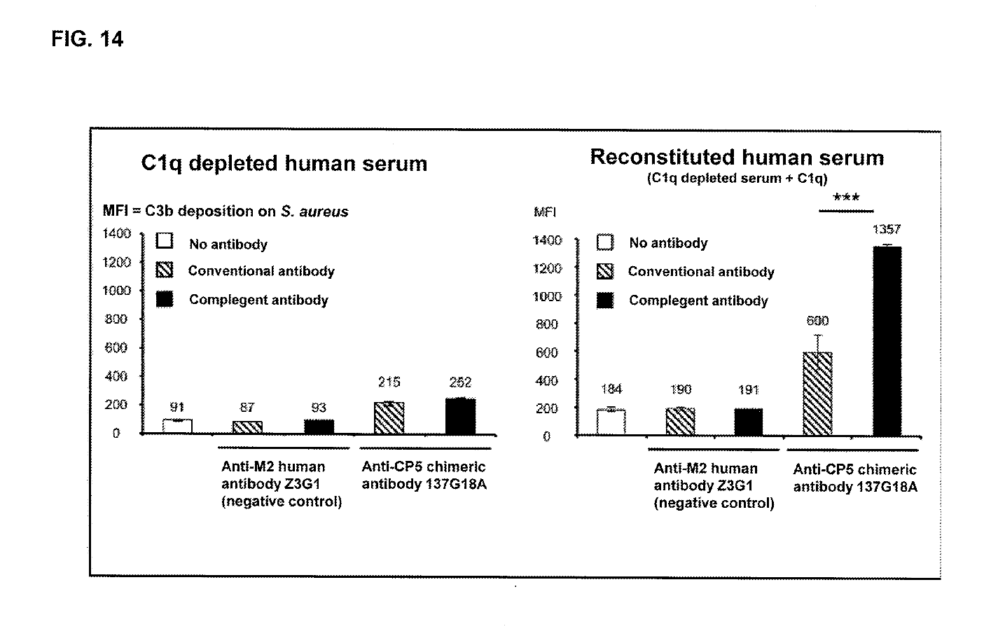

Further, FIG. 14 represents the results of the examination of the effect of anti-CP5 antibody on complement C3b deposition on Staphylococcus aureus Lowenstein in the presence or absence of C1q. As the experimental condition, the antibody was used in 500 μg, and 10⁸ CFU of bacteria were used. Serum concentration was 2.5%.

As shown in FIG. 14, the C3b deposition on bacterial surface was canceled by depletion of complement C1q from the serum, but was recovered by addition of C1q protein. The anti-CP5 chimeric antibody-mediated deposition of C3b on bacterial surface was thus found to be C1q dependent.

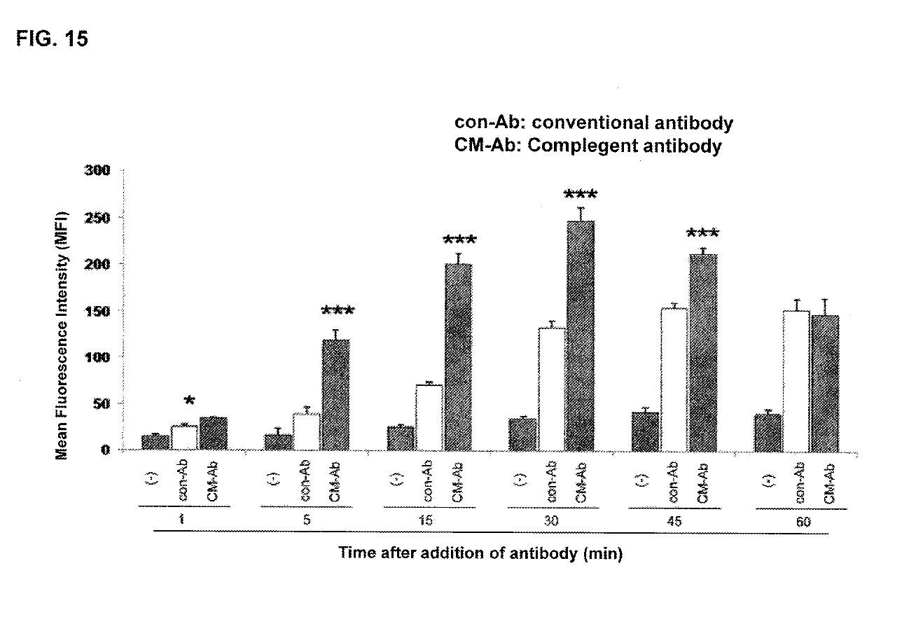

The anti-CP5 chimeric antibody-mediated C3b deposition on bacterial surface was assayed over a time course. As the assay method, 5×10⁸ CFU/mL (10⁸ CFU total) of Staphylococcus aureus strain Lowenstein was reacted in 2.5% human serum-supplemented RPMI in the presence or absence of 1.25 mg/mL (total amount 0.25 mg) of anti-CP5 antibody. C3b deposition indicative of complement deposition on bacteria was then measured by FACS analysis using FITC-labeled anti-C3b antibody. Experiment was conducted in triplicate. Average values and standard deviation of the obtained results are shown in the graph of FIG. 15. (*, p<0.05; ***, p<0.0005; two-tailed unpaired t-test.)

As a result, it was found that C3b deposition on bacteria had a peak within 60 min after the addition of the antibody, as shown in FIG. 15.

### Example 6

### Evaluation of Anti-CP5 Antibody-Mediated Phagocytosis in the Presence of Human Polymorphonuclear Neutrophils

The phagocytosis mediated by anti-CP5 chimeric antibody 137G18A against Staphylococcus aureus strain Loewenstein in the presence of human polymorphonuclear neutrophils was assayed in the same manner as in Example 2.

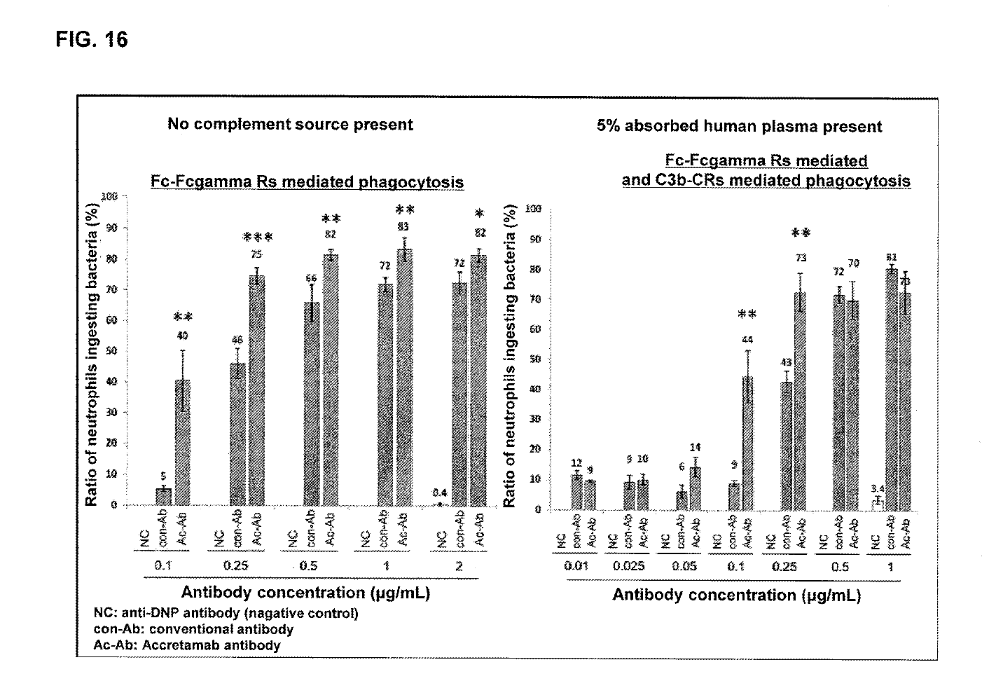

As a result, as shown in FIG. 16, Ac-137G18A-mediated phagocytosis was higher than that of con-137G18A in the presence of human polymorphonuclear neutrophils, with or without the human complement. The Ac-137G18A-mediated phagocytosis through both FcγRIII and complement receptor was thus found to be high.

### Example 7

### Evaluation of Anti-PspA Chimeric Antibody 140H1-Mediated Phagocytosis Activity against Streptococcus pneumoniae Strain

An anti-Streptococcus pneumonial surface protein A (hereinafter, referred to as PspA) mouse antibodies were established from mice immunized with a recombinant PspA derived from Streptococcus pneumoniae strains D39 and TIGR4.

The anti-PspA monoclonal antibodies 140H1 and 140G1 were reconstructed to anti-PspA chimeric antibodies 140H1 and 140G1 by using an ordinary method. The nucleotide sequence and the amino acid sequence of VH of anti-PspA monoclonal antibody 140H1 are represented by SEQ ID NOs: 7 and 8, respectively. The nucleotide sequence and the amino acid sequence of VL, of anti-PspA monoclonal antibody 140H1 are represented by SEQ ID NOs: 9 and 10, respectively. The nucleotide sequence and the amino acid sequence of VH of anti-PspA monoclonal antibody 140G1 are represented by SEQ ID NOs: 11 and 12, respectively. The nucleotide sequence and the amino acid sequence of VL of anti-PspA monoclonal antibody 140G1 are represented by SEQ ID NOS: 13 and 14, respectively. The anti-PspA chimeric antibodies 140H1 and 140G1 were used as samples in the following forms. IgG antibodies con-140H1 and con-140G1 with core fucose bound to the complex type N-linked sugar chain attached to Fc of antibody; PT-140H1 antibody and PT-140G1 antibody without core fucose bound to the complex type N-linked sugar chain attached to Fc of antibody; NCM-140H1 antibody and NCM-140G1 antibody with enhanced CDC by amino acid residue substitution in the Fc region; and NAc-140H1 antibody and NAc-140G1 antibody without core fucose bound to the complex type N-linked sugar chain attached to Fc of antibody and with enhanced CDC by amino acid residue substitution in the Fc region.

NCM-140H1, NCM-140G1, NAc-140H1, and NAc-140G1 were constructed according to the method described in the specification of US Patent Application Publication 2012/0010387. The amino acid sequence of the Fc region is represented by SEQ ID NO: 2.

In each well of 96-well plates, 2.5×10⁶ fluorescein-labeled Streptococcus pneumoniae cells were reacted for 30 min with 5×10⁵ human polymorphonuclear neutrophils suspended in 200 μL HBSS/PBS and 10 μg/mL (final concentration) of anti-DNP antibody (isotype control), PT-140H1 or NAc-140H1.

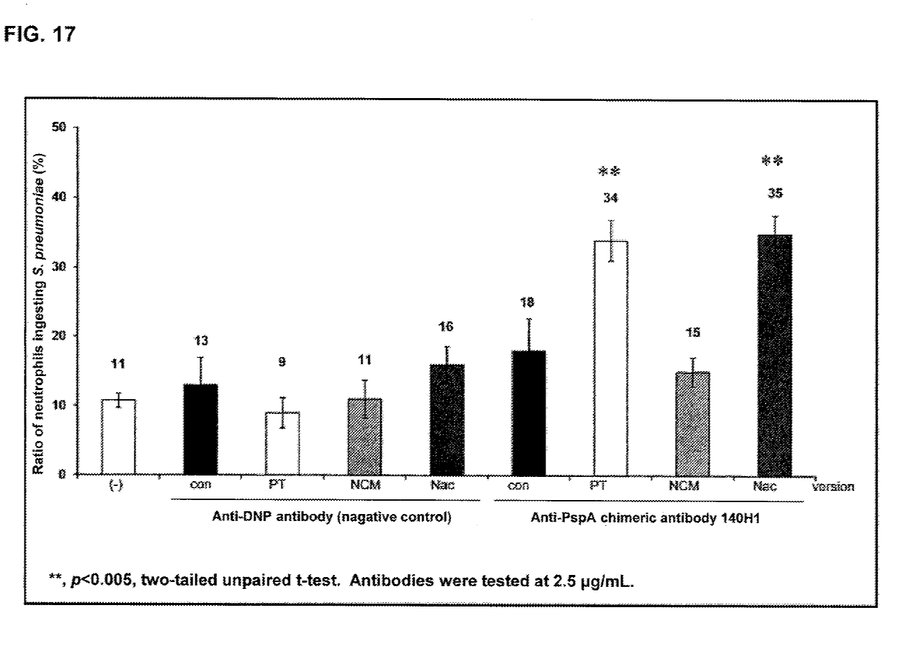

Subsequently, the reaction solution was washed, and the ratio of human polymorphonuclear neutrophils that phagocytized Streptococcus pneumoniae in 200 viable human polymorphonuclear neutrophils per sample was measured by fluorescence microscopy. Ethidium bromide with a final concentration of 0.25 mg/mL was added to the samples to differentiate bacteria ingested into human polymorphonuclear neutrophils and extracellular bacteria. Experiment was conducted in quadruplicate. The average values and the standard deviation of one representative result from at least two experiments are shown in the graph of FIG. 17. **, p<0.005; ***, p<0.0005 vs. con-140H1 two-tailed unpaired t-test.

As a result, as shown in FIG. 17, the ingestion of Streptococcus pneumoniae strain D39 by human polymorphonuclear neutrophils in the absence of the complement was increased by the addition of PT-140H1 and NAc-140H1, compared to the addition of con-140H1 and NCM-140H1. The result showed that the complement-independent opsonophagocytosis mediated by anti-PspA antibody was heavily dependent on the antibody's fucosylation level.

### Example 8

### Evaluation of Anti-PspA Chimeric Antibody 140H1- and 140G1-Mediated Complement Deposition on Streptococcus pneumoniae Strain Surface

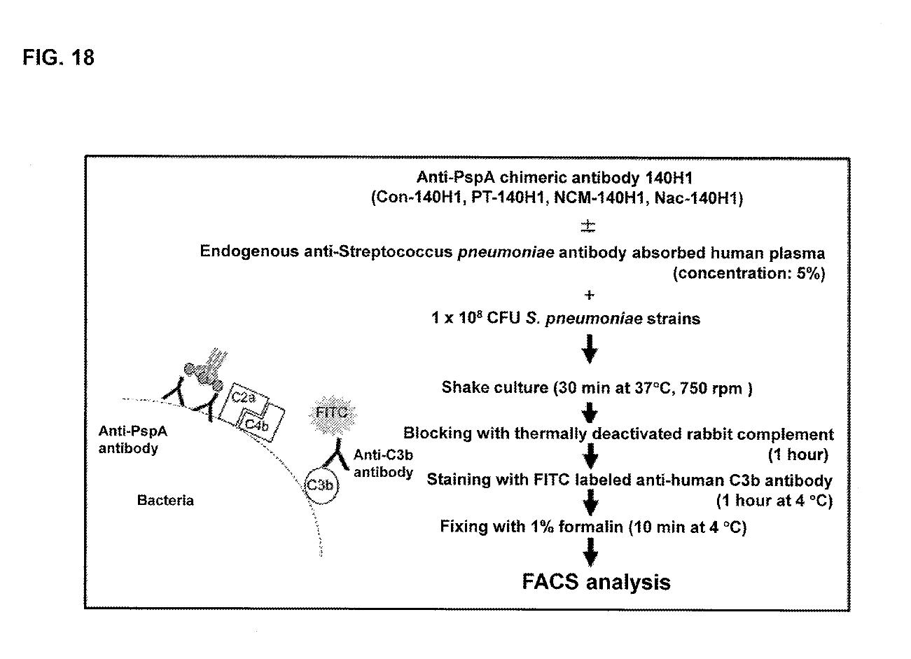

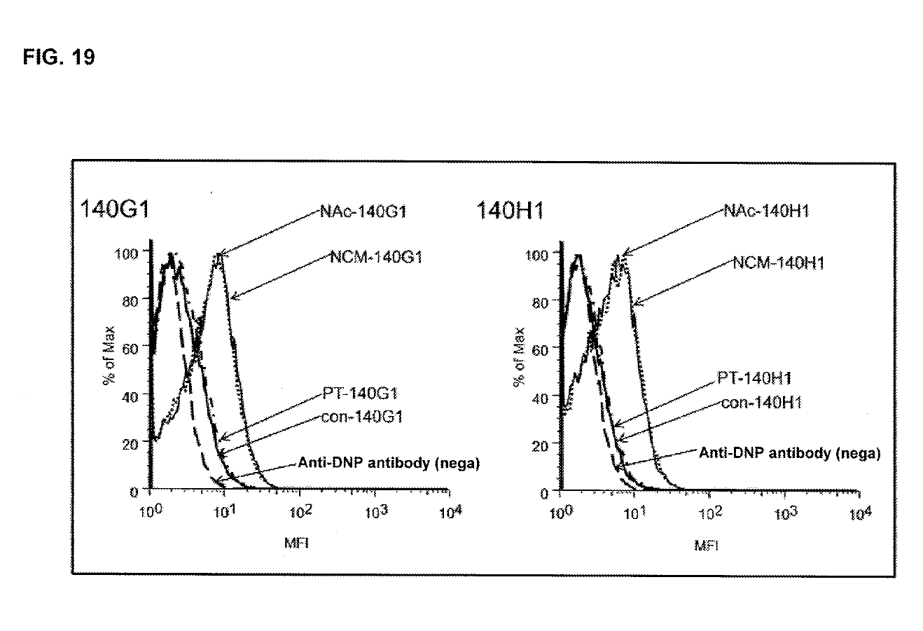

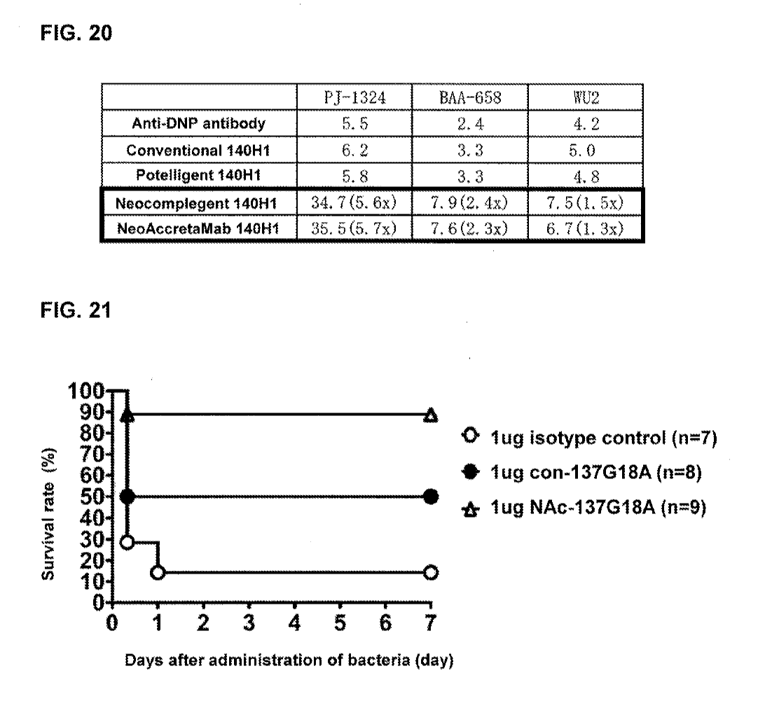

The C3b deposition activity mediated by anti-PspA chimeric antibodies 140H1 and 140G1 for bacterial surface in the presence of human serum was assayed with three Streptococcus pneumoniae strains WU2, BAA-658, and PJ-1324 by using the method represented in FIG. 18. FIG. 19 shows histograms representing the results of the experiment using BAA-658. Further, the results of the measurement using the three strains are summarized in FIG. 20.

As a result, as shown in FIGS. 19 and 20, NCM-140H1 and NAc-140H1 had increased C3b deposition activity than con-140H1 and PT-140H in the presence of human complement in all of the three Streptococcus pneumoniae strains. Similar results were obtained for anti-PspA chimeric antibody 140G1.

The modified antibody with increased CDC activity, such as Complegent and Accretamab, were found to have potential to enhance the C3b complement receptor-mediated phagocytosis induced by anti-PspA antibodies.

### Example 9

### In Vivo Medicinal Efficacy Evaluation of Anti-CP5 Chimeric Antibody in RNU Rat Sepsis Model

To evaluate whether the Fc modification of antibody can enhance the in vivo medicinal efficacy of anti-CP5 chimeric antibody, the in vivo medicinal efficacy of con-137G18A and NAc-137G18A was compared using a rat systemic infection model.

The in vivo medicinal efficacy of con-137G18A and NAc-137G18A was evaluated with a Rowett Nude (hereinafter, abbreviated to RNU) rat model administered with bacteria preopsonized with 1 μg of each antibody in vitro. Staphylococcus aureus strain MSSA Reynolds (ATCC-25923) was used as bacteria.

As shown in FIG. 21, RNU rats infected with MSSA Reynolds preopsonized with NAc-137G18A had a higher survival rate than RNU rats that were infected with MSSA Reynolds preopsonized with either anti-DNP antibody used as an isotype control or con-137G18A.

From these results, NAc-137G18A was found to be potentially more protective against infection than con-137G18A in vivo.

Although the present invention has been described in connection with the specific embodiments in detail, it will be apparent to those skilled in the art that various modifications and changes may be made thereto without departing from the scope and spirit of the present invention. Meanwhile, this application is based on U.S. Provisional Application No. 61/682,404 filed on Aug. 13, 2012, the entire contents of which are incorporated hereinto by reference.

### FREE TEXT OF SEQUENCE LISTING

SEQ ID NO. 1: Amino acid sequence of IgG1/IgG3 chimeric Fc

SEQ ID NO. 2: Amino acid sequence of IgG1/IgG3 chimeric Fc_N392K

1. A method for increasing deposition of complement C3b on the bacterial surface and phagocytosis by phagocytes using an antibody binding to a molecule on the bacterial surface, wherein the antibody is modified by substituting one or more amino acid residues with other amino acids so as to show more enhanced complement-dependent cytotoxicity (hereinafter, abbreviated to CDC) than the antibody before substitution of the amino acid residues.

2. The method according to claim 1, wherein one or more amino acid residues are included in the CH2 domain of the antibody Fc region.

3. The method according to claim 1, wherein the modified antibody shows more enhanced complement C1q-binding activity than the antibody having an amino acid sequence before substitution of the amino acid residue.

4. The method according to claim 1, wherein the subclass of the antibody binding to a molecule on the bacterial surface is human IgG1.

5. The method according to claim 1, wherein the bacteria are one or more bacteria selected from Gram-positive and Gram-negative bacteria.

6. The method according to claim 5, wherein the Gram-positive bacteria are one or more Gram-positive bacteria selected from Bacillus, Listeria, Staphylococcus, Streptococcus, Enterococcus, Clostridium, and Mycobacterium.

7. The method according to claim 5, wherein the Gram-negative bacteria are one or more Gram-negative bacteria selected from Pseudomonas, Escherichia, Salmonella and Acinetobacter.

8. The method according to claim 1, wherein the molecule on the bacterial surface is one or more molecules selected from ganglioside, capsular polysaccharide (CP), surface protein (SP) and lipopolysaccharide (LPS).

9. The method according to claim 1, wherein the modified antibody has a complex type N-linked sugar chain in the Fc region, and 20% or more of the total complex type N-linked sugar chain binding to the Fc region is the sugar chain having no α1,6-fucose bound to N-acetylglucosamine at the reducing end of the sugar chain.

10. A pharmaceutical composition for bacterial infections, comprising a pharmaceutically acceptable carrier and an antibody binding to a molecule on the bacterial surface, which is modified by substituting one or more amino acid residues with other amino acids so as to show more enhanced CDC than the antibody before substitution of the amino acid residues as an active agent.

11. A therapeutic method for bacterial infections, characterized in that an antibody binding to a molecule on the bacterial surface, which is modified by substituting one or more amino acid residues with other amino acids so as to show more enhanced CDC than the antibody before substitution of the amino acid residues, is used to increase deposition of complement C3b on the bacterial surface and phagocytosis by phagocytes, thereby reducing bacterial proliferation.


SEQUENCE LISTING (14 sequences)

SEQ ID NO: 1 - protein, 217 aa, Artificial
  Description of the artificail sequence Synthetic IgG1/IgG3 chimeric Fc amino acid sequence
APELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNHYTQKSLSLSPGK

SEQ ID NO: 2 - protein, 217 aa, Artificial
  Description of the artificail sequence Synthetic IgG1/IgG3 chimeric Fc_N392K amino acid sequence
APELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYKTTPPMLDSDGSFFLYSKLTVDKSRWQQGNIFSCSVMHEALHNHYTQKSLSLSPGK

SEQ ID NO: 3 - DNA, 354 bp, Mus musculus
  CDS (1)..(354)
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatgcagctttcatatccagactgagcatcagcaaggacaattccaagagccaagttttctttaaaatgaacagtctgcaagctaatgacacagccatatattactgtgccagagacaggtacgacgtcagggcctttgactactggggccaaggcaccactctcacagtctcctct

SEQ ID NO: 4 - protein, 118 aa, Mus musculus
QVQLKQSGPGLVQPSQSLSITCTVSGFSLTNYGVHWVRQSPGKGLEWLGVIWSGGNTDYNAAFISRLSISKDNSKSQVFFKMNSLQANDTAIYYCARDRYDVRAFDYWGQGTTLTVSS

SEQ ID NO: 5 - DNA, 321 bp, Mus musculus
  CDS (1)..(321)
gacattgtgatgacccagtctcaaaaattcatgtccacatcagtaggagacagggtcagcatcacctgcaaggccagtcagaatgttcgtactgctgtagcctggtatcaacagaaaccagggcagtctcctaaagcactgatttacttggcatccaaccggcacactggagtccctgatcgcttcacaggcagtggatctgggacagatttcactctcaccattagcaatgtgcaatctgaagacctggcagattatttctgtctgcaacattggaattatctgtacacgttcggaggggggaccaagctggaaataaaa

SEQ ID NO: 6 - protein, 107 aa, Mus musculus
DIVMTQSQKFMSTSVGDRVSITCKASQNVRTAVAWYQQKPGQSPKALIYLASNRHTGVPDRFTGSGSGTDFTLTISNVQSEDLADYFCLQHWNYLYTFGGGTKLEIK

SEQ ID NO: 7 - DNA, 354 bp, Mus musculus
  CDS (1)..(354)
cagatccagttggtgcagtctggacctgaactgaagaagcctggagagacagtcaggatctcctgcaaggcttctggatataccttcacaactgctggaatgcagtgggtacaaaaggtgccgggaaagggtttgcagtggattggctggataaatacccactctggagtgccaacatatgcagaggacttcaagggacgtattgccttctctttggaaacctctgccagcgctgcatatttacagataagcaacctcaaagatgaggacacggctacgtatttctgtgggagatccgggacgggatatgctatggactattggggtcaaggaacctctgtcatcgtctcctca

SEQ ID NO: 8 - protein, 118 aa, Mus musculus
QIQLVQSGPELKKPGETVRISCKASGYTFTTAGMQWVQKVPGKGLQWIGWINTHSGVPTYAEDFKGRIAFSLETSASAAYLQISNLKDEDTATYFCGRSGTGYAMDYWGQGTSVIVSS

SEQ ID NO: 9 - DNA, 339 bp, Mus musculus
  CDS (1)..(339)
gacactgtgatgtcacagtctccatcctccctagtcgtgtcagttggagagaaggttagtatgagctgcaagtccagtcagaaccttttatatactaacaatcaaaagaactacttggcctggtaccagcagaaaccaggacagtctcctaaactactgatttactgggcatccactcgggaatctggggtccctgatcgctttacaggcagtggatctgggacagatttcactctcaccatcaccagtgtgaaggctgaagacctggcagtttattattgtcagcaatattacacctatccatacacgttcggaggggggaccaagctggaaataaaa

SEQ ID NO: 10 - protein, 113 aa, Mus musculus
DTVMSQSPSSLVVSVGEKVSMSCKSSQNLLYTNNQKNYLAWYQQKPGQSPKLLIYWASTRESGVPDRFTGSGSGTDFTLTITSVKAEDLAVYYCQQYYTYPYTFGGGTKLEIK

SEQ ID NO: 11 - DNA, 348 bp, Mus musculus
  CDS (1)..(348)
caggtgcagctgaaggagtcaggacctggcctggtggcgccctcacagagcctgtccatcacatgcaccgtctcagggttctcattaatcggctatggtgtaaactgggttcgccagcctccaggaaagggtctggagtggctgggaatgatatggggtgatggaagcacagactataatttagttctcaaatccagactgagcatcaacaaggacaactccaagagccaagttttcttgcaaatgaacagtctgcaaactgatgacacagccaggtactactgtgccagaggccctcaatgggggtttgcttactggggccaagggactctggtcagtgtctctgca

SEQ ID NO: 12 - protein, 116 aa, Mus musculus
QVQLKESGPGLVAPSQSLSITCTVSGFSLIGYGVNWVRQPPGKGLEWLGMIWGDGSTDYNLVLKSRLSINKDNSKSQVFLQMNSLQTDDTARYYCARGPQWGFAYWGQGTLVSVSA

SEQ ID NO: 13 - DNA, 336 bp, Mus musculus
  CDS (1)..(336)
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttataatcttccgacgttcggtggaggcaccaagctggaaatcaaa

SEQ ID NO: 14 - protein, 112 aa, Mus musculus
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLPTFGGGTKLEIK